(19) United States
(12) Patent Application Publication
(10) Pub. No.: US 2012/0091221 A1
(43) Pub. Date: Apr. 19, 2012

(54) Wick Diffuser

(75) Inventors: Kylie L. Levake, Racine, WI (US); Christopher S. Hoppe, Milwaukee, WI (US); Jason L. Halonen, Racine, WI (US); Anne E. Louly, Racine, WI (US); Maciej K. Tasz, Racine, WI (US); Steven V. Bisbikis, Hawthorn Woods, IL (US); Ronald M. Llanes, Lisle, IL (US); Daniel J. Neidlinger, Bartlett, IL (US)

(73) Assignee: S.C. JOHNSON & SON, INC.

(21) Appl. No.: 13/224,442

(22) Filed: Sep. 2, 2011

Related U.S. Application Data
(60) Provisional application No. 61/380,893, filed on Sep. 8, 2010.

(51) Int. Cl. A61L 9/04

(52) U.S. Cl. 239/44

(57) ABSTRACT

A wick diffuser employing a cellulosic based wick is disclosed. The wick diffuser includes a base from which first and second arcuate panels upwardly and outwardly extend and flanking a center cellulosic wick. The wick is substantially planar in shape and upwardly extends from a container of volatile organic compound housed within the base. The base may be manufactured from a polymeric material or include a polymeric insert provided within a wooden surround. The diffuser has numerous spill resistance features as well as a low insertion force needed by the user to install the wick.

### CROSS-REFERENCE TO RELATED APPLICATIONS

This is a non-provisional U.S. patent application, which claims priority under 35 U.S.C. §119(e) to U.S. Provisional Patent Application Ser. No. 61/380,893 filed on Sep. 8, 2010, the entirety of which is incorporated by reference herein.

### FIELD OF THE DISCLOSURE

The present disclosure generally relates to diffusers for releasing fragrance or other volatile organic compounds, and more particularly relates to diffusers using a planar wicking device to do so.

### BACKGROUND OF THE DISCLOSURE

Diffusers are well known. Such devices commonly are used to release fragrance into the ambient air of a living space or the like to provide a pleasing aroma. In addition to fragrance, the diffusers can be used to release other volatile organic compounds (VOC) as well, as including, but not limited to, deodorizers and pesticides. With many diffusers, a source of fragrance or other VOC is provided within a cartridge, with the cartridge itself being provided relative to a heating device. Upon electrical activation of the heating device, the fragrance is heated so as to vaporize same and thus disseminate the fragrance into the ambient air. To facilitate such dissemination, it is also common to use such devices as fans or other air flow devices, piezoelectric actuators, and oscillators. The electrical actuation can be provided by battery or AC power such as a well known “plug-in” diffusers as marketed by the present assignee under its Glade® trademark.

In other diffusers, both, electrical and non-electrical, a wicking device may be used. With such devices, a supply of liquid fragrance or other VOCs is provided within a container into which, and from which, a wicking device, such as a cylindrical porous media, outwardly extends. By way of capillary action, the liquid fragrance or VOC is drawn up the wick away from the container and into the outer extremities of the wicking device. The fragrance, pesticide, or other VOC is then disseminated into the ambient air either by simple evaporation, or can be facilitated again through the use of devices such as the fans, heating devices, oscillators and piezoelectric actuators as indicated above.

While effective, it would be desirable to manufacture the wick from materials other than sintered plastic and in end shapes other than cylindrical rods. In addition, it would be desirable to manufacture the diffuser such that the wick is maintained in a clean, unsaturated manner until activation by the user is desired. In addition, it would be desirable to provide such a diffuser with increased spill resistance and wick retention once activated. Finally, it would be desirable to provide such a diffuser with increased aesthetic appeal and decreased environmental impact.

### SUMMARY OF THE DISCLOSURE

In accordance with at least one aspect of the disclosure, a wick diffuser is therefore provided having a base from which first and second arcuate panels upwardly extend. A supply of liquid VOC is provided within a container and set within the base. A wick holder extends upwardly from the container of VOC, and retains a planar shaped wick manufactured from cellulosic materials. The wick includes an elongated stem surrounded by a protective shroud or wick holder adapted to penetrate a plug seal provided within the container.

In at least some other aspects of the disclosure, a wick diffuser having a base, at least one panel, the panel receivable within the base and a container assembly positionable within the base and housing a liquid therewithin is provided. The wick diffuser can also include a wick securable to the container assembly for dispersing fragrance from the liquid housed within the container assembly into a surrounding area, the wick being substantially planar in shape and including a body portion and a stem portion.

In some other aspects of the disclosure, a wick diffuser having a base, a container assembly positionable within the base is provided. The wick diffuser can also include a wick and a shroud assembly having a pair of wick holders and a shroud for securing the wick in position, the shroud assembly securable within the container assembly. The wick diffuser can additionally include a pair of arcuate panel members securable within the base and extending upward and away from the base.

In yet other aspects of the disclosure, a wick diffuser kit is provided. The wick diffuser kit can include a first piece having a base and a pair of panel members and a refill having (a) a container assembly having a bottle housing a liquid and a plug for preventing access of the liquid by a user; (b) a shroud assembly having a pair of wick holders and a shroud, the shroud assembly capable of being assembled to the container assembly; and (c) a wick capable of being secured to the shroud assembly, the refill capable of being used with the first piece.

These and other features and aspects of the disclosure will be better understood upon reading the following detailed description when taken in conjunction with the accompanying drawings.

### BRIEF DESCRIPTION OF THE DRAWINGS

For a more complete understanding of the disclosed methods and apparatus, reference should be made to the embodiments illustrated in greater detail on the accompanying drawings, wherein:

FIG. 1 is a perspective view of a first embodiment of a wick diffuser constructed in accordance with the teachings of the disclosure;

FIG. 2 is a top view of the wick diffuser of FIG. 1;

FIG. 3 is a perspective view of a base of the first embodiment depicted in FIG. 1;

FIG. 4 is a perspective view of the VOC container and wick holder of FIG. 1;

FIG. 5 is a front view of the wick of FIG. 1;

FIG. 6 is a cross-sectional view taken along line 6-6 of FIG. 1;

FIG. 7 is a cross-sectional view taken along line 7-7 of FIG. 1;

FIG. 8 is a bottom perspective view of the wick holder and puncture plug;

FIG. 9 is a cross-sectional view of the wick holder and puncture plug entering the VOC container;

FIG. 10 is a cross-sectional view of the wick holder and puncture plug after puncturing the seal provided within the VOC container;

FIG. 11 is a perspective view of an alternative embodiment of the VOC container with a threaded cap mounted thereto;

FIG. 12 is a cross-sectional view taken along line 12-12 of FIG. 11;

FIGS. 13a-c depict an alternative embodiment of the wick including folding flaps for sealing and retention purposes;

FIG. 14 is a perspective view of another alternative embodiment of a wick including a folding flap for sealing and retention purposes;

FIG. 15 is yet another alternative embodiment of a wick having folded flaps;

FIG. 16 is a perspective view of a second embodiment of a wick diffuser including a wooden base;

FIG. 17 is a perspective view of the wooden base of FIG. 16;

FIG. 18 is a perspective view of the plastic insert of the wooden embodiment of FIG. 16;

FIG. 19 is a perspective view of the assembled diffuser having the plastic insert provided within the wooded base;

FIG. 20 is a cross-sectional view taken along line 20-20 of FIG. 16;

FIG. 21 is a cross-sectional view taken along line 21-21 of FIG. 16;

FIG. 22 is a cross-sectional view taken along line 22-22 of FIG. 16;

FIG. 23 is a perspective view of a third alternative embodiment of a wick diffuser constructed in accordance with the teachings of the disclosure and including lighting capability;

FIG. 24 is a top view of the lighted wick diffuser of FIG. 23;

FIGS. 25 and 26 are perspective views of fourth and fifth embodiments, respectively, of a wick diffuser constructed in accordance with the teachings of the present disclosure;

FIG. 27 is an exploded view of the wick diffuser of FIG. 25 showing a base, a container assembly, a shroud assembly, a wick and a pair of arcuate panels, in accordance with at least some embodiments of the present disclosure;

FIG. 28 is a perspective view of a first embodiment of the base of FIG. 27, in accordance with at least some embodiments of the present disclosure;

FIG. 29 is a top view showing the container assembly disposed within the base of FIG. 28;

FIG. 30 shows a perspective view, in cut-away, of a portion of the container assembly disposed within the base of FIG. 28;

FIG. 31 shows a top view of a second embodiment of the base of FIG. 27, in accordance with at least some other embodiments of the present disclosure;

FIG. 32 shows, in cut-away, a portion of the container assembly disposed within the base of FIG. 31;

FIG. 33 is a bottom view of the bases of FIGS. 28 and 31;

FIG. 34 shows an exploded view of a third embodiment of the base of FIG. 27, in accordance with yet other embodiments of the present disclosure;

FIG. 35 shows a bottom perspective view of a part of the base of FIG. 34;

FIG. 36 shows a first embodiment of one of the arcuate panels of FIG. 27 in greater detail, in accordance with at least some embodiments of the present disclosure;

FIG. 37 shows a perspective view of a first embodiment of a proper installation of the arcuate panel of FIG. 36 with respect to the base of FIG. 27;

FIG. 38 shows, in cut-away, a portion of FIG. 37 in greater detail;

FIGS. 39 and 40 show perspective views of an improper installation of the arcuate panel of FIG. 36 with respect to the base of FIG. 27;

FIG. 41 shows a second embodiment of one of the arcuate panels of FIG. 27 in greater detail, in accordance with at least some other embodiments of the present disclosure;

FIG. 42 shows, in cut-away, a portion of a fourth embodiment of the base of FIG. 27 for receiving the arcuate panel of FIG. 41;

FIG. 43 shows a perspective view of a proper installation of the arcuate panel of FIG. 41 with respect to the base of FIG. 42;

FIG. 44 shows, in cut-away, a portion of FIG. 43;

FIGS. 45 and 46 show perspective and cut-away views, respectively of improper installation of the arcuate panel of FIG. 41 with respect to the base of FIG. 42;

FIG. 47 shows a perspective view of a first embodiment of the container assembly of FIG. 27, in accordance with at least some embodiments of the present disclosure;

FIG. 48 shows an exploded view of the container assembly of FIG. 47;

FIG. 49 shows a front view of the container assembly of FIG. 47;

FIG. 50 shows a cross-sectional view taken alone line 50-50 of FIG. 49;

FIG. 51 shows a portion of the cross-sectional view of FIG. 50;

FIG. 52 shows a front view, showing additional features of the container assembly of FIG. 47;

FIGS. 53a-c show, in cut-away, portions of the cross-sectional view of FIG. 51 illustrating yet additional features of the container assembly of FIG. 47;

FIG. 54 shows an exploded view of a second embodiment of the container assembly of FIG. 27, in accordance with some other embodiments of the present disclosure;

FIG. 55 shows an assembled view of the container assembly of FIG. 54;

FIG. 56 shows a perspective view of the wick of FIG. 27, in accordance with at least some embodiments of the present disclosure;

FIGS. 57a and 57b show perspective views of a first part of the shroud assembly of FIG. 27;

FIG. 57c shows a front view of the first part of the shroud assembly of FIGS. 57a and 57b;

FIG. 58 shows, in cut-away, a portion of the wick of FIG. 56 installed with respect to the first part of the shroud assembly of FIGS. 57a-c;

FIG. 59 shows a perspective view of a second part of the shroud assembly of FIG. 27, in accordance with at least some embodiments of the present disclosure;

FIG. 60 is a cross sectional view of the container assembly and the shroud assembly, taken along lines 60-60 of FIG. 62b below;

FIG. 61 is a perspective view showing the wick of FIG. 56 installed within the shroud assembly of FIG. 27;

FIG. 62a is an exploded view showing the wick and shroud assembly of FIG. 61 for installation within the container assembly;

FIG. 62b is a perspective installed view of FIG. 62a;

FIG. 63 is a perspective view, in cut-away, of a portion of FIG. 62b;

FIG. 63a shows a portion of FIG. 63 in cut-away;

FIG. 64 shows another portion of FIG. 63 in cut-away; and

FIG. 65 shows a starter kit, in accordance with some teachings of the present disclosure.

While the following detailed description has been given and will be provided with respect to certain specific embodiments, it is to be understood that the scope of the disclosure should not be limited to such embodiments, but that same are provided simply for enablement and best mode purposes. The breadth and spirit of the present disclosure is broader than the embodiments specifically disclosed and encompassed within the claims eventually appended hereto.

### DETAILED DESCRIPTION

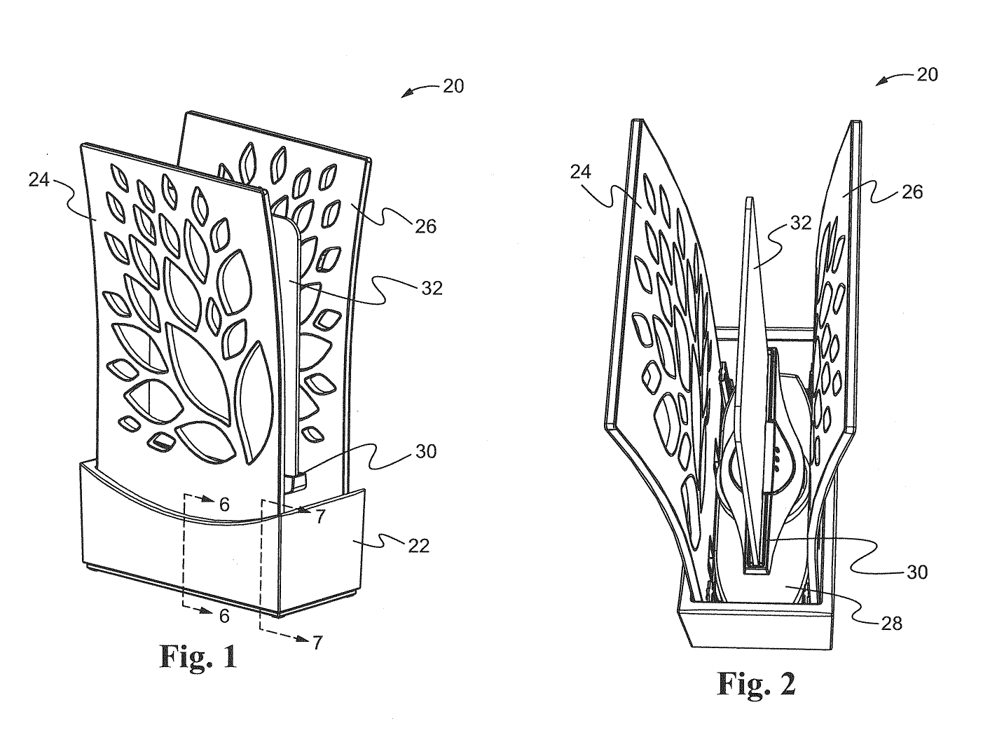

Referring now to the drawings, and with specific reference to FIG. 1, a wick diffuser constructed in accordance to the teachings of the disclosure is generally referred to by reference numeral 20. Such a diffuser has particular applicability in disseminating fragrance or other volatile organic compounds (VOC) into an ambient air environment without the use of self-contained electrical actuators, fans, or the like. Moreover, as will be described in further detail herein, by manufacturing components of the diffuser from natural materials, the diffuser is partially bio-degradable and environmentally friendly.

The wick diffuser 20 in the embodiment depicted in FIG. 1, includes a base 22 from which first and second arcuate panels 24, 26 upwardly extend. In addition, as shown best in FIG. 2, the wick diffuser 20 includes a container 28 filled with the volatile organic compound (VOC) and from which upwardly extends a wick holder 30, and from which itself extends a wick 32. As will be further defined herein, the arcuate panels 24, 26 provide decoration and also protect the wick 32. For example, if the diffuser 20 is tipped over, the panels 24, 26 will touch the surface on which the diffuser 20 is resting, so that the saturated wick 32 does not come into direct contact with the surface. The panels 24, 26 may have holes in them and the opne area may be sized to allow substantial air to flow normal to the wick 32, thereby promoting VOC dispersion.

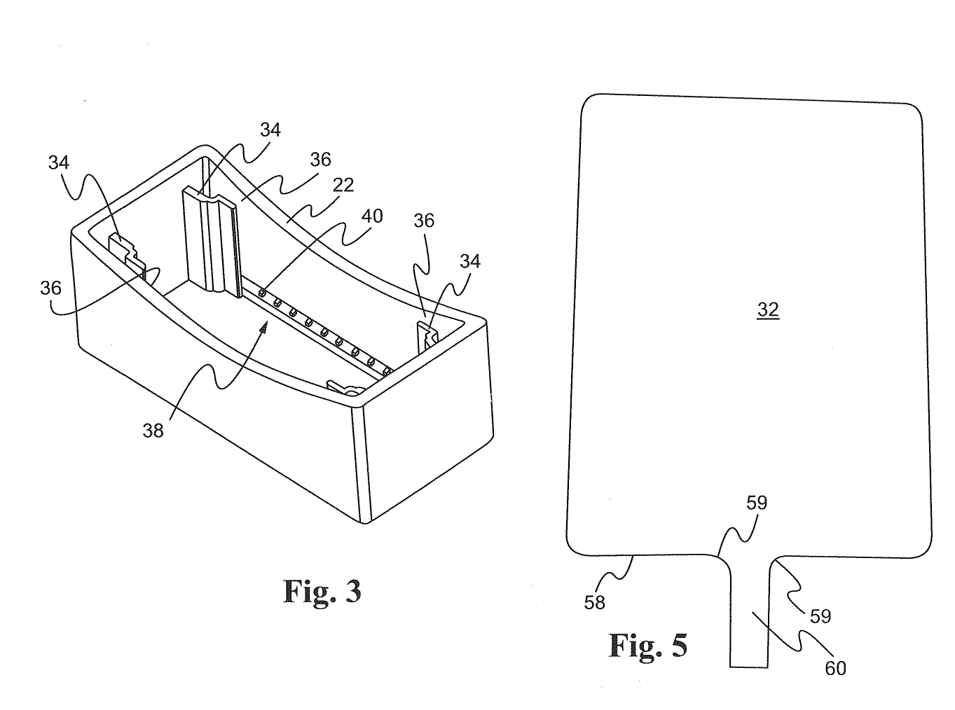

As will be noted in FIG. 2, the container 28 is dimensioned relative to the interior dimensions of the base 22 so as to be easily received therein and removed therefrom. The first and second panels 24, 26 are received within the base 22 either through the use of an interference fit alone or in combination with one or more proactive retention features as well. For example, the panels 24, 26 may have a slip fit within the base 22 so that the user may remove the panels 24, 26 if they like. Spring fingers or other proactive retention features may be used to retain the panels 24, 26 so that they are not loose. As shown best in FIG. 3, those retention features could include a plurality of wall supports 34 extending inwardly from the base 22 so as to define receiving slots 36 dimensioned so as to frictionally receive the first and second arcuate panels 24, 26. In addition, the base 22 may include an internal wall 38 against which the first and second panels 24, 26 bottom out and which themselves may include a plurality of friction nubs 40 to facilitate retention of the panels 24, 26 therein. In alternative embodiments, not shown, the base 22 may include inwardly directed detents adapted to be received in apertures provided within the first and second panels 24, 26. In such an embodiment, the panels 24, 26 would not be easily be removed and thus could be viewed as a more permanent fixture as opposed to the embodiment depicted in FIGS. 1-3.

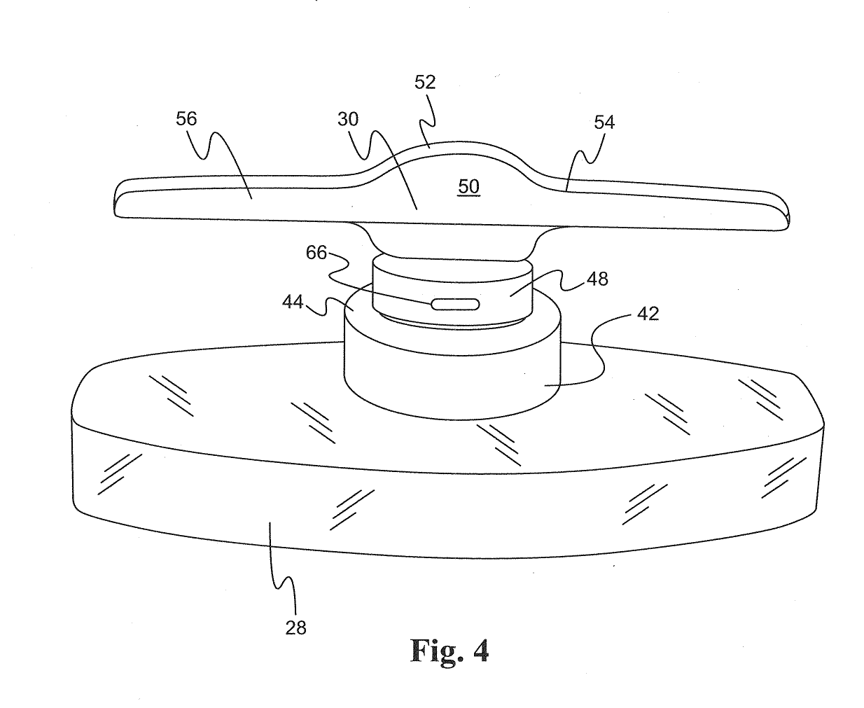

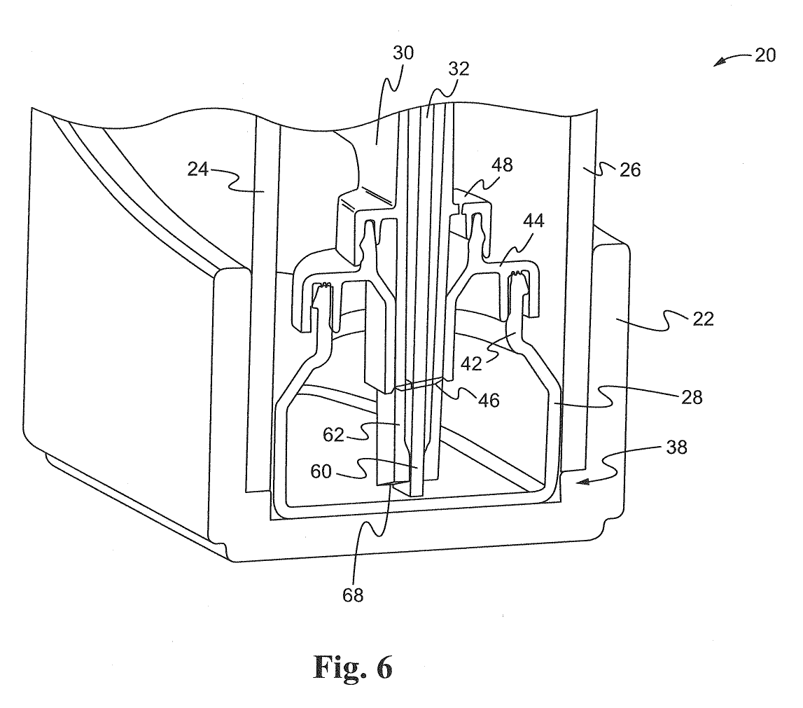

Turning now to FIG. 4, the container 28 and wick holder 30 is shown in more detail. More specifically, the container 28 includes a relatively low profile so as to be fully received within the base 22. The container 28, as further shown in FIG. 6, includes an upwardly extending neck 42 against which a sealing plug is frictionally received. The sealing plug 44 includes a ruptureable membrane 46 which temporarily seals the volatile organic compound within the container 28 until use by a consumer is desired as will be described in further detail herein. Referring back to FIG. 4, it can be seen that the wick holder 30 then upwardly extends from the sealing plug 44. More specifically, the wick holder 30 includes a mounting ring 48 from which upwardly extends clamping panels 50, 52. The clamping panels 50, 52 are spaced apart appropriately by way of a receiving recess 54 so as to frictionally grip the wick 32 when inserted therein. In addition, the wick holder 30 may include a plurality of inwardly directed teeth (not shown) to hold the wick 32 in a few key places via an interference fit. By holding the wick 32 in a few key places, fragrance migration over the wick holder 32 is limited. Laterally extending from the clamping panels 50, 52 is a drip gutter 56. The drip gutter 56 is adapted to receive any downwardly directed volatile organic compound which either drips or is pulled by way of gravity from the wick 32. In so doing the drip gutter 56 both recycles the volatile organic compound for further use and also retains the clean aesthetic appeal of the diffuser 20.

Referring now to FIG. 5, the wick 32 is shown in further detail to be, in the depicted embodiment, planar in side profile and rectangular in front detail. Downwardly extending from a bottom edge 58 is a stem 60. Fillets 59 may be provided at the transition from the bottom edge 58 to the stem 60 to increase the strength of the stem 60. It is the stem 60 which penetrates the aforementioned membrane 46 when use is desired. While not wanting to be tied to any particular theory, in one particular embodiment, it has been found by the inventors that a stem having a width less than ten percent of the overall width of the wick 32 is desirable to draw the volatile organic compound into the wick to both facilitate wicking and diffusion. In that particular embodiment, the less than ten percent ratio may equate to a stem having a width of approximately eight millimeters and the wick itself having an overall width of seventy-six millimeters, but again this is simply exemplary of one embodiment with other dimensions certainly being possible. With respect to the material from which the wick 32 is manufactured, any cellulosic or sintered material is possible including those directly derived from wood, balsa, or the like, such as paper. Moreover, as will be discussed in further detail herein, the wick 32 may be die-cut from a larger sheet stock of such material.

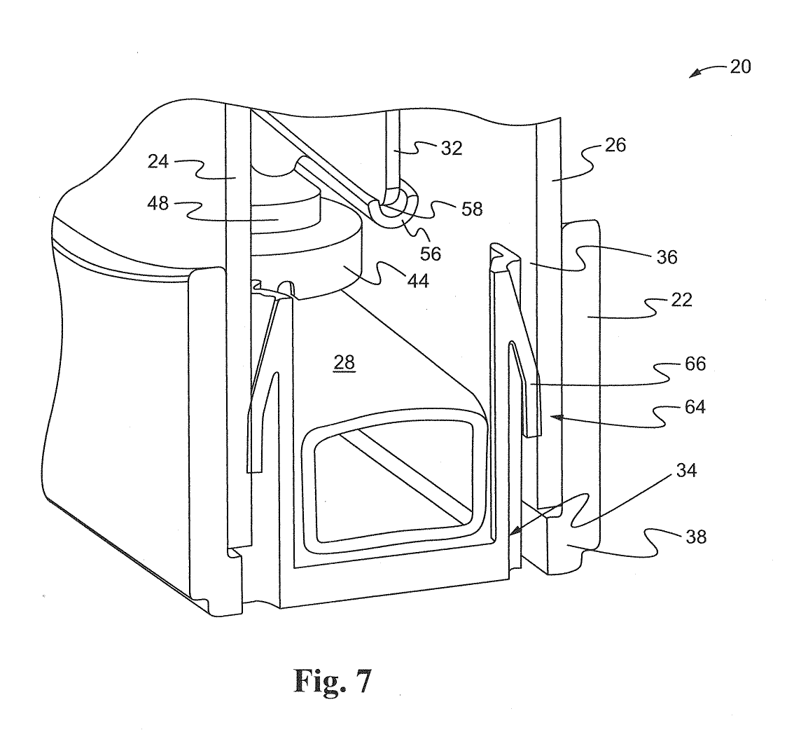

When assembled, the components fit together as depicted in FIGS. 6-7. Starting with FIG. 6, it will be noted that the first and second arcuate panels 24, 26 are frictionally received within the base 22 and bottom out against the internal walls 38. The internal walls 38 create a fragrance well inside the base 22 to prevent any possible leakage of the VOC from passing through any holes in the floor of the base. In addition, it will be noted that the exterior surface of the container 28 is received within interior surface of the first and second arcuate panels 24, 26. While not shown, the base 22, walls 38 and/or the container 28 may include one or more keys or interlocks to ensure that only appropriately manufactured refill containers are used with such a base.

FIGS. 6 and 7 also show that the wick 32 is fully received within the container when the wick holder has been mounted atop the sealing plug 44. Moreover, it will be noted that the wick holder 32 includes a downwardly depending protective shroud 62. The shroud 62 protects the cellulosic wick 32 as it penetrates the membrane 46. Without the use of the protective shroud 62 the wick 32 may not be sufficiently strong to penetrate the membrane 46 or, if it does have sufficient strength, may be required to be manufactured from an undesirably thick dimension. In addition, as will be noted from FIG. 7, the base 22 includes the aforementioned wall support 34 to frictionally receive the first and second arcuate panels 24, 26. The wall support 34 may include an outwardly biased retention arm 64 having cam surfaces 66 to guide the first and second arcuate panels 24, 26 into the receiving slots 36 and then, once received, to bias the panels 24, 26 against the base 22.

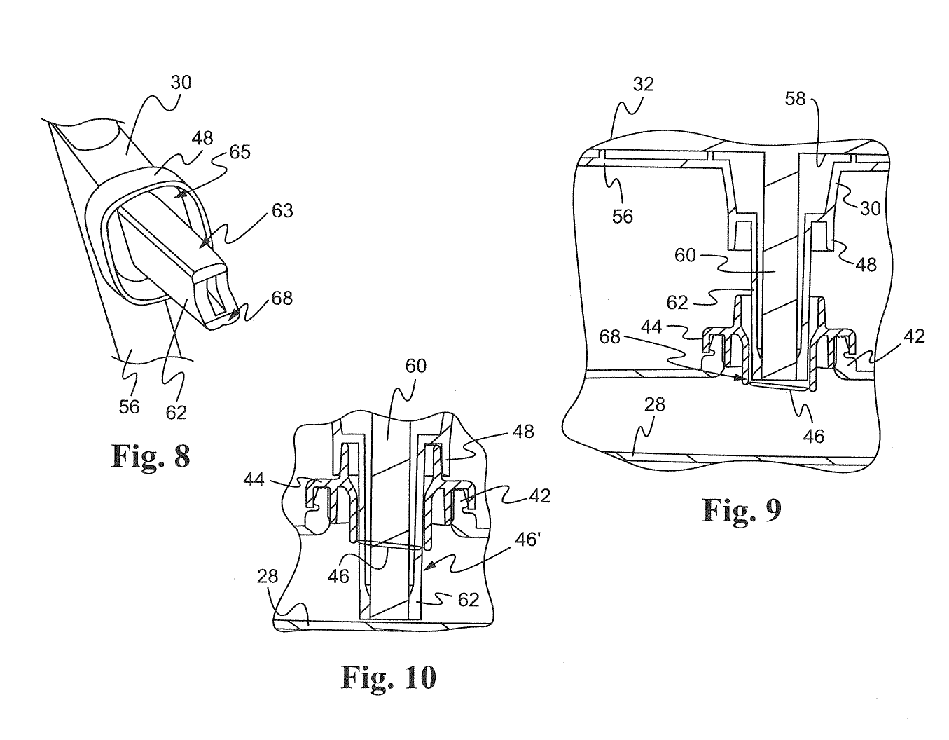

In order to facilitate puncturing the membrane 46, it will be noted that the protective shroud 62, as shown best in FIGS. 8-10, includes a bottom surface 68 provided at a compound angle. In so doing, a single point of entry is established and thus the insertion force required for the wick 32 to puncture the membrane 46 is lowered. This is best depicted in the cross-sectional views of FIGS. 9 and 10. Starting with FIG. 9, it will be noted that the protective shroud 62 and rupturable membrane 46 are not provided so as to engage each other along parallel lines, or planes, but rather against a single point of entry. Either the bottom surface 68 can be provided with the compound angle as indicated with respect to FIG. 8, or as shown in FIGS. 9 and 10, the membrane itself can be provided at such an angle. In the case of the latter, depicted in FIGS. 9 and 10, this can be accomplished by manufacturing the membrane 46 in a process known as “coining” wherein a scored line or a line of relative weakness is provided around the perimeter between the membrane 46 and the sealing plug 44. Accordingly, when the user downwardly presses the protective shroud 62 against the sealing membrane 46, the membrane or coin is ruptured thus causing the remainder of the membrane 46 or coin to downwardly depend from the sealing plug in a hanging fashion best depicted in phantom line by reference numeral 46′ in FIG. 10. The sealing membrane or coin 46 is dimensioned so as to not engage the lower surface of the container 28 even after being ruptured as shown in FIG. 10.

Referring now to FIG. 8, it can be seen that the protector shroud 62 includes a vent channel 63 extending along the longitudinal axis of the protector shroud 62. Vent channel 63 cooperates with a venting hole 65 provided in the wick holder 30 so as to continually provide an air flow path to the outside ambient environment thus facilitating wicking of the volatile organic compound into wick 32. Mounting ring 48 may also include a detent 66 to prevent a user from being able to disassemble the wick holder 30 from the plug 44 once the product is activated. This limits user access to the VOC formulation as a safety benefit.

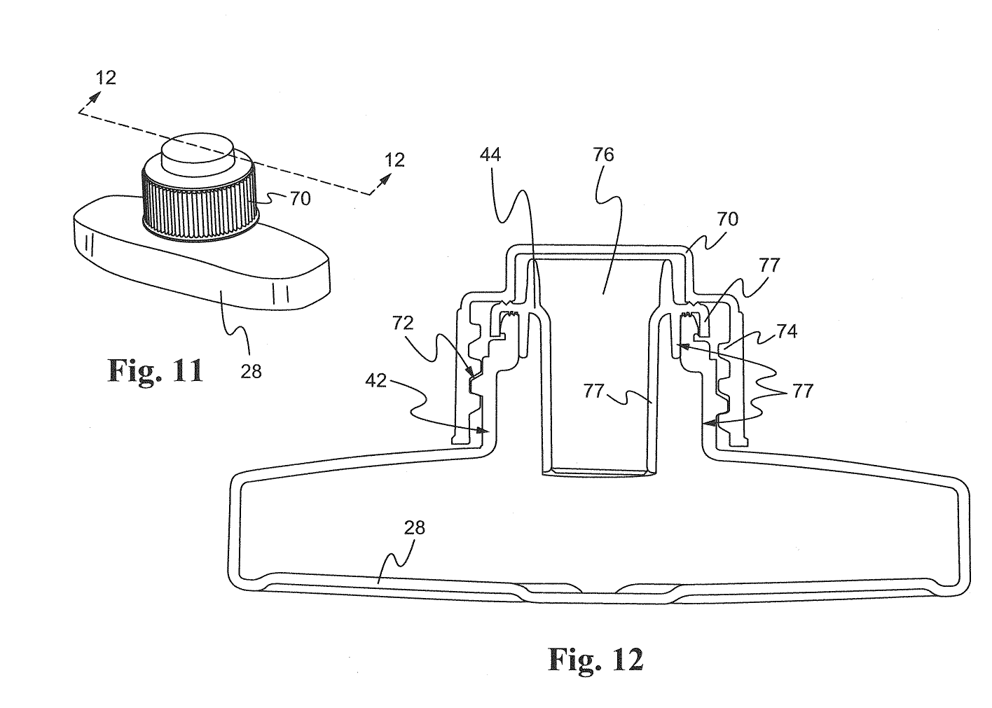

In an alternative embodiment, the sealing membrane or coin 46 could be combined with a threaded cap 70 to facilitate the sealing function until it is desired by a user to activate the wick diffuser 20. More specifically, the neck 42 of the container 28 can be provided with a plurality of external threads 72 with the cap 70 itself including a plurality of internal threads 74 such that, upon rotation, the two can be temporarily joined together as depicted in FIGS. 11 and 12. When desired, the user can counter-rotate the cap 70 to thus provide an opening 76 into which the stem 60 of the wick 32 can be inserted.

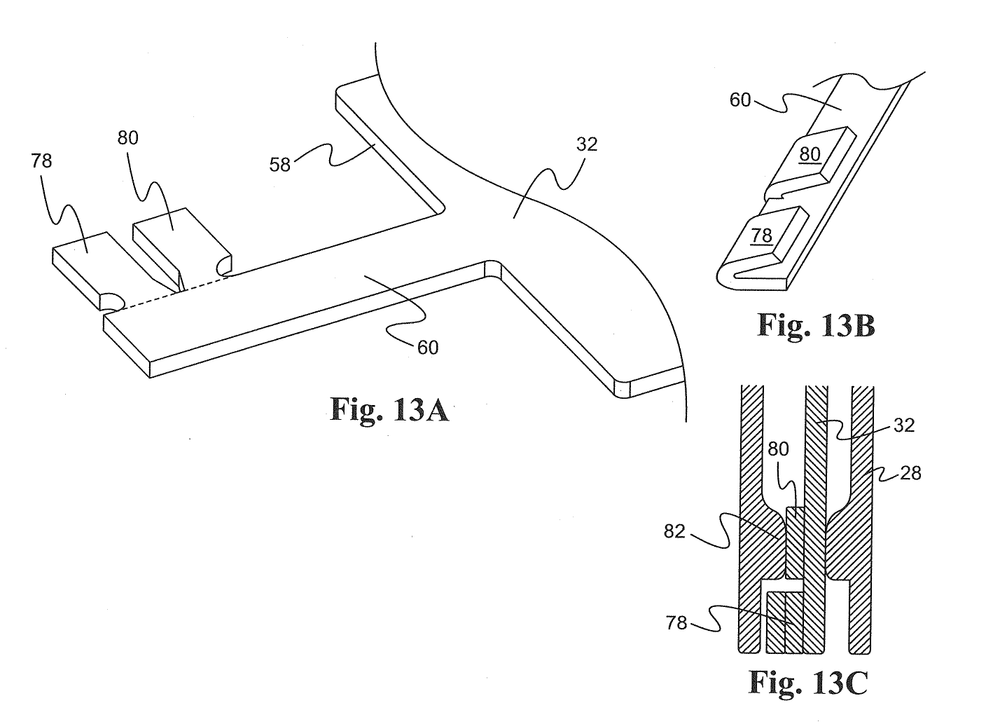

In order to seal the wick 32 within the container 28 and provide enhanced spill resistance should the wick diffuser 22 be inadvertently toppled, either the container and/or sealing plug 44 can be manufactured with multiple barrier walls 77 as depicted in the foregoing figures, or, the wick 32 can itself include a plurality of sealing features. Starting with FIG. 13, one embodiment would provide the stem 60 with first and second folding flaps labeled as bottom flap 78 and top flap 80. In a comparison between FIGS. 13a and b, it can be seen that the wick 32 can be die-cut in the orientation shown in FIG. 13a with the top and bottom flaps 78, 80 being foldable into the configuration shown in FIG. 13b. Once folded into the configuration of FIG. 13b, the wick 32 can be inserted into the container 28 with the container 28 itself including a reduced diameter venturi 82. Once fully inserted, the bottom flap 78 will not be retained in a completely folded configuration by the venturi 82, but rather will be allowed to be biased outwardly as shown best in FIG. 13c. However, the top flap 80 will, conversely, be held tightly together in a closed configuration by the venturi walls 82. In so doing, the top flap 82 in conjunction with the venturi 82 concurrently seal the wick 32 within the bottle 28, with the bottom flap 78 serving a wick retention function in that if the wick 32 is pulled in an upward direction relative to FIG. 13c, the bottom flap 78 will engage the venturi 82 and prevent such upward motion.

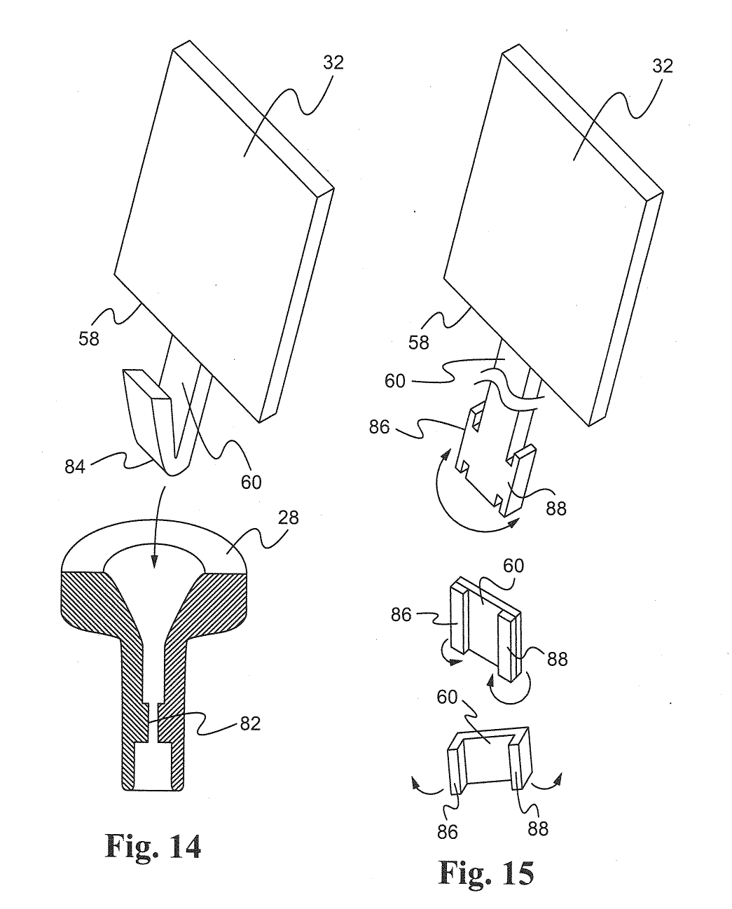

FIGS. 14 and 15 are further embodiments along the same lines of FIG. 13a-c. Starting with FIG. 14, it includes a single flap but one folded along a horizontal bottom surface 84. Once inserted, the bottom flap would engage venturi 82 within the sealing plug. In the embodiment of FIG. 15, first and second flaps are again provided, but this time in the form of a left flap 86 and a right flap 88.

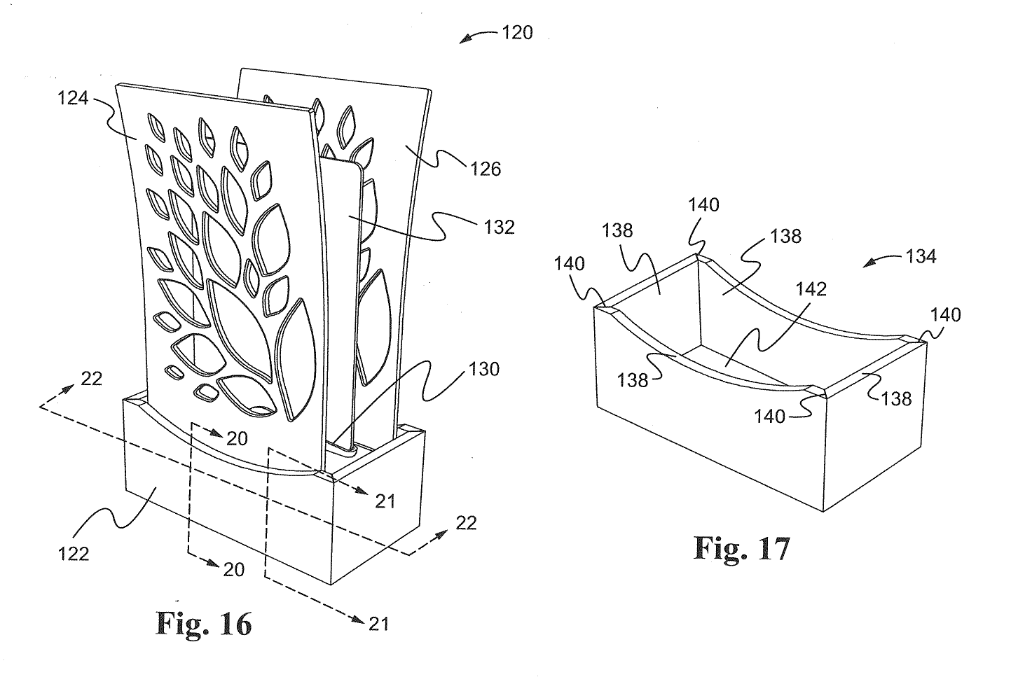

The foregoing embodiment is with respect to a base 22 manufactured from a polymeric material such as, but not limited to polyethylene terephthalate (PET), glass fiber and mineral filled polypropylene. In the second embodiment depicted in FIG. 16, the exterior of the base 22 is manufactured from wood and thus provides a substantially different aesthetic appeal. As shown therein, the wick diffuser of the second embodiment is generally referred to by reference numeral 120. It again includes a base 122 from which first and second arcuate panels 124, 126 upwardly extend. In addition, again a wick 132 upwardly extends from a container 128 of volatile organic compound and sits within a wick holder 130. However, in order to retain the container 128 within the base 122 and still provide the wooden surround 134, a plastic insert 136 is utilized.

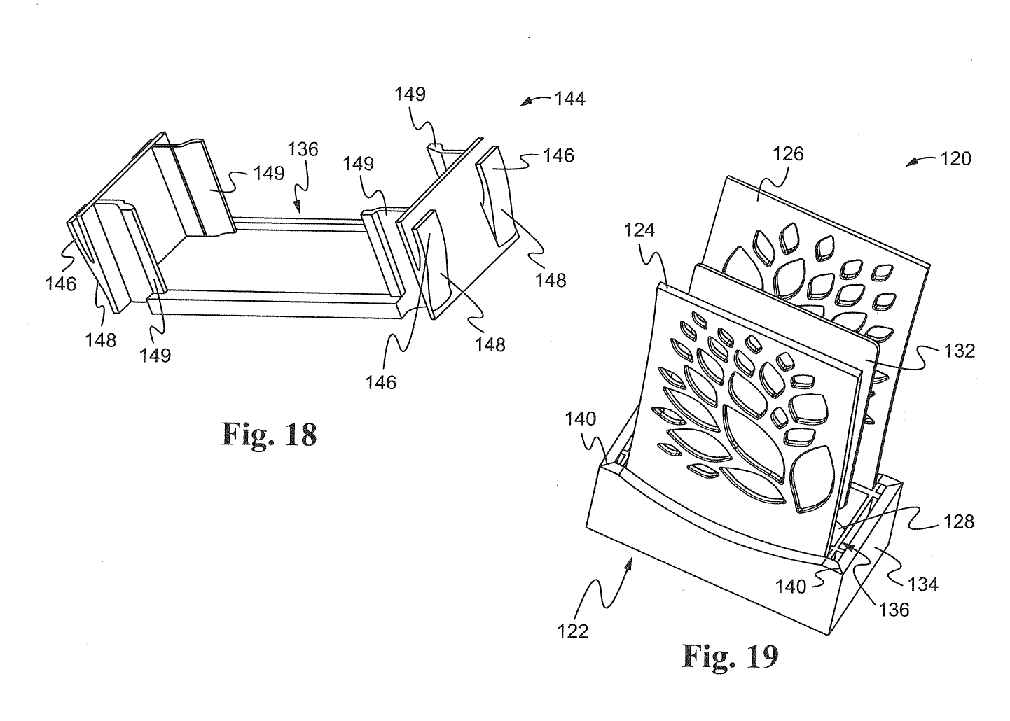

As shown best in a comparison between FIGS. 17, 18, and 19, the wooden surround 134 essentially forms a five-sided enclosure having four side walls 138 joined at mitered corners 140 and having a bottom wall 142. The plastic insert 136 is frictionally received within the wooden surround 134 and includes a plurality of retention features 144 to facilitate and accentuate such a frictional fit. More specifically, the retentions features 144 may include outwardly biased arms 146, each of which include cam surfaces 148 to more easily allow the plastic insert 136 to first be received within the wooden surround 34 and then be retained therein. In addition, wall supports 149 are provided to bias the panels toward the wood surround 134 when inserted in a manner similar to the first embodiment to facilitated an interference fit.

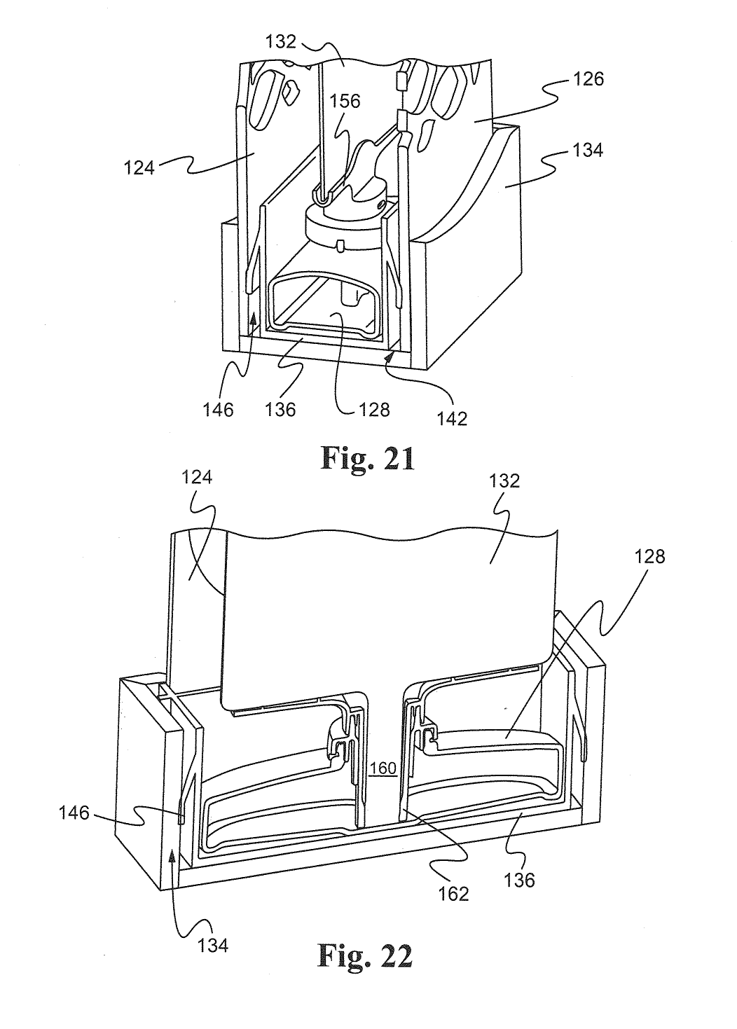

Once assembled, it can be seen from FIGS. 21-23 that the arcuate panels 124, 126 are frictionally received within the wooden surround 134 and sit atop a bottom ledge 150 of the plastic insert 136. In turn, the container 128 is dimensioned so as to be frictionally received within the interior surface provided by the arcuate panels 124, 126. Moreover, as shown best is FIGS. 21 and 22, the retention features 144, specifically the biasing arms 146, frictionally engage the end wall 138 of the wooden surround to frictionally maintain the plastic insert 136 into the wooden surround 134.

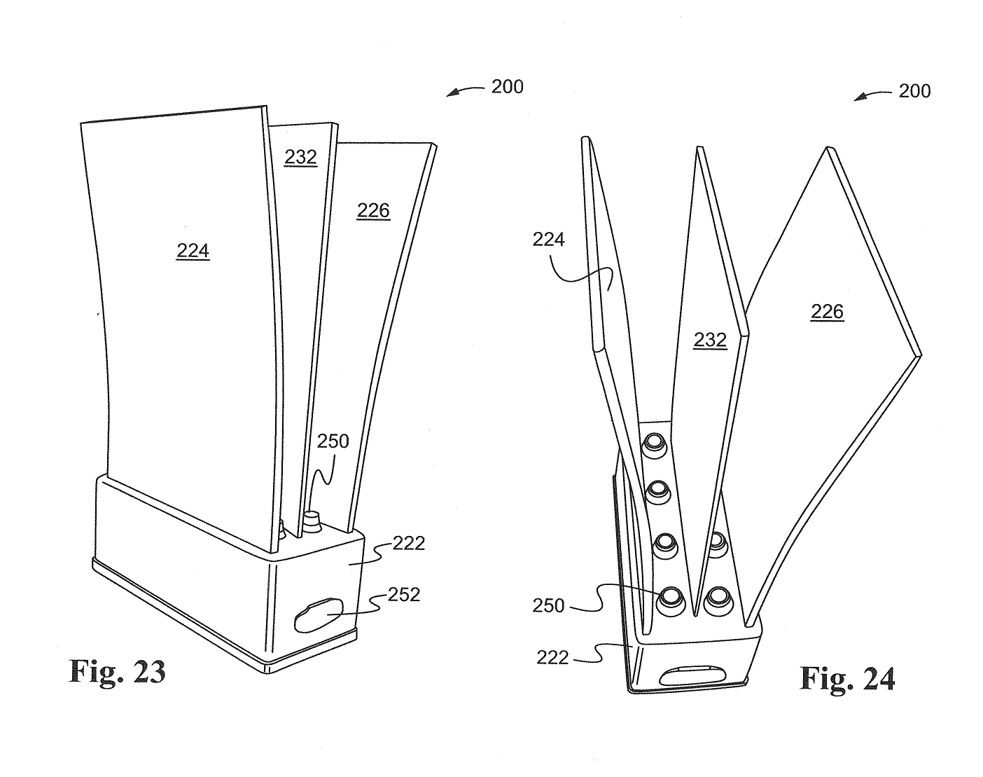

Referring now to FIGS. 23 and 24, another embodiment of the present disclosure is set forth and generally referred to by reference numerals 200. Again the wick diffuser 220 includes a base 222 as well as first and second arcuate side panels 224, 226 and an interior wick 232. However, the wick diffuser 220 of this embodiment further includes lighting elements 250 adapted to illuminate the side panels 224, 226 as well as the wick 232. In order to accentuate the use of light in such an embodiment, the side panels 224, 226 can be manufactured from a translucent material such as glass or acrylic. In addition, a power module 252 such as a battery is mounted within the base 22 to energize the lights 250. The lighting elements may include any known lighting technology including, but not limited to, light emitting diodes (LEDs), incandescent, and fluorescent.

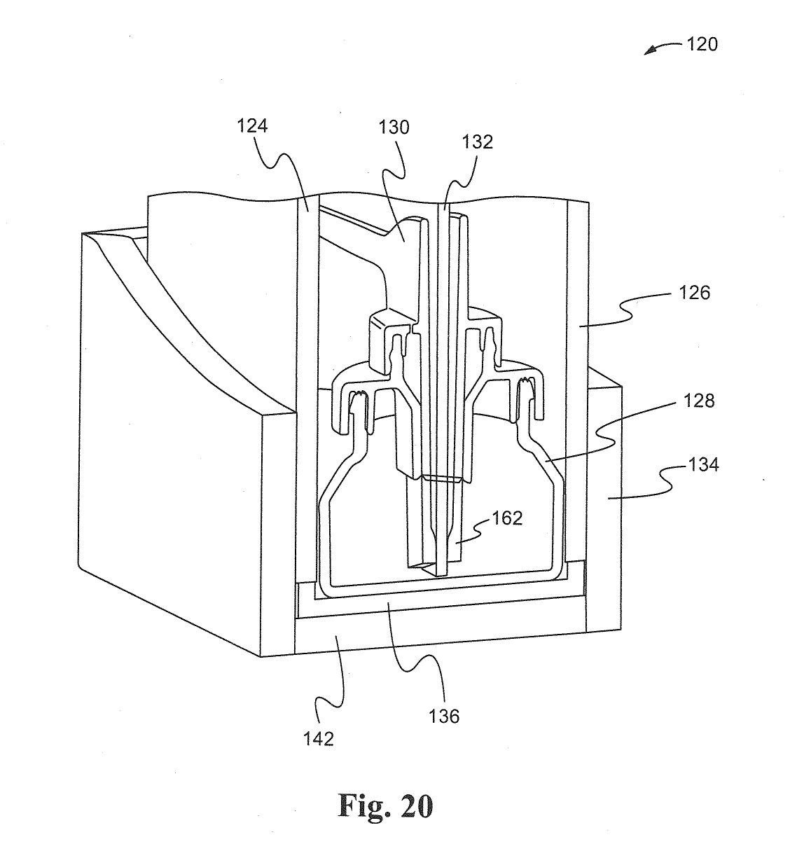

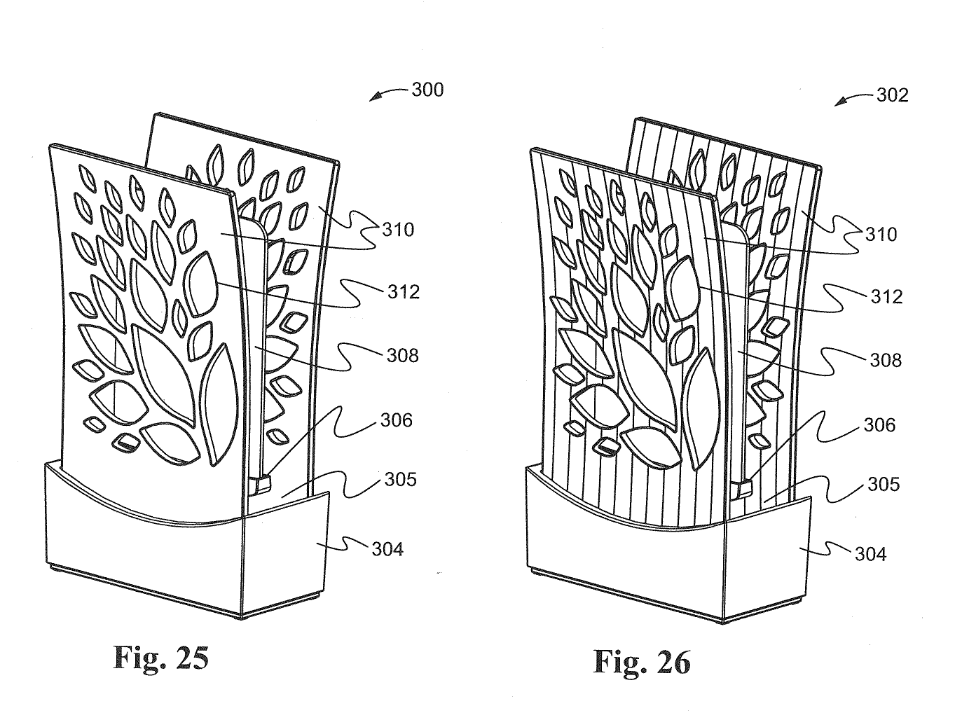

Referring now to FIGS. 25 and 26, fourth and fifth embodiments, respectively, of wick diffusers 300 and 302 are shown, in accordance with at least some embodiments of the present disclosure. Similar to the previous embodiments of FIGS. 1-24, the embodiments of FIGS. 25 and 26 can each include a base 304, a container assembly 305, a shroud assembly 306, a wick 308 and a pair of arcuate panels 310 assembled together in operational association.

With respect to the arcuate panels 310 in particular, they extend upwardly and outwardly from the base 304 to provide aesthetic appeal to the wick diffusers 300, 302, and protection to the wick 308. Each of the pair of panels 310 can be constructed from various materials, such as plastic, to impart a smooth feel to the panels, as the panels of the wick diffuser 300, or they can be constructed from wood (e.g., bamboo) with a wood grain, such as, the panels of the wick diffuser 302. In some embodiments, the pair of panels 310 constructed out of plastic may be filled with minerals to add mass to the wick diffusers 300 and 302 and, also to impart to the panels a ceramic feel. In other embodiments, the plastic pair of panels 310 may be filled with natural fibers like grain hull or wood pulp to mimic the feel of wood panels. In yet other embodiments, one or both of the pair of panels 310 can be constructed of materials other than those described above.

Furthermore, each of the arcuate panels 310 can be designed with several cut-out patterns, such as, a pattern 312, on its surface to impart air flow to the wick 308. In at least some embodiments, the cut-out openings of the pattern 312 may occupy about thirty three percent (33%) of the entire panel area of each of the arcuate panels 310 to provide an optimal fragrance evaporation rate. In other embodiments, the pattern 312 and the percentage of the cut-out openings in the pattern 312 of the arcuate panels 310 may vary depending upon the rate of fragrance evaporation and air flow desired. Furthermore, the pattern 312 can be designed such that the pattern is centered from the left, right and top edges of the pair of panels 310 and in at least some embodiments, a different pattern can be employed for each of the pair of arcuate panels 310.

In addition, to avoid breaking or cracking of the pair of panels 310 during construction, the pattern 312 can be designed such that thickness of the panels between the various cut-outs is three (3) millimeters. In other embodiments, the thickness of the pair of panels 310 between various cut-outs can vary depending upon the pattern employed, the material used for making the panels, as well as the construction technique employed for constructing the panels.

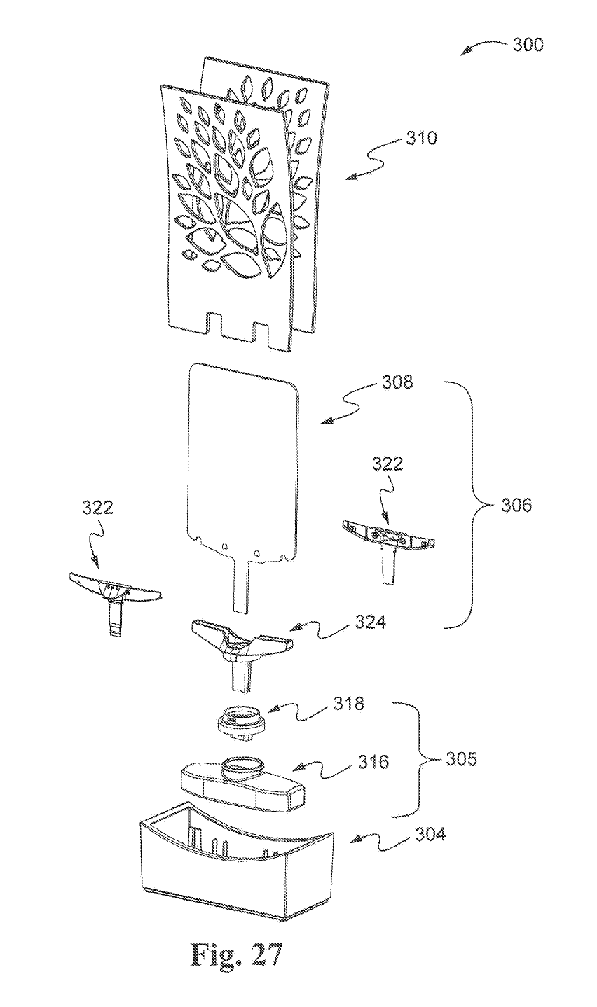

Referring now to FIG. 27, an exploded view of the wick diffuser 300 is shown, in accordance with at least some embodiments of the present disclosure. It will be understood that although the present disclosure is described with respect to the wick diffuser 300, the same teachings will be applicable to the wick diffuser 302 as well. As shown, the exploded view can include the base 304, the container assembly 305, the shroud assembly 306, the wick 308 and the pair of panels 310. Each of the aforementioned elements of the wick diffuser 300 is described in greater detail in the following figures.

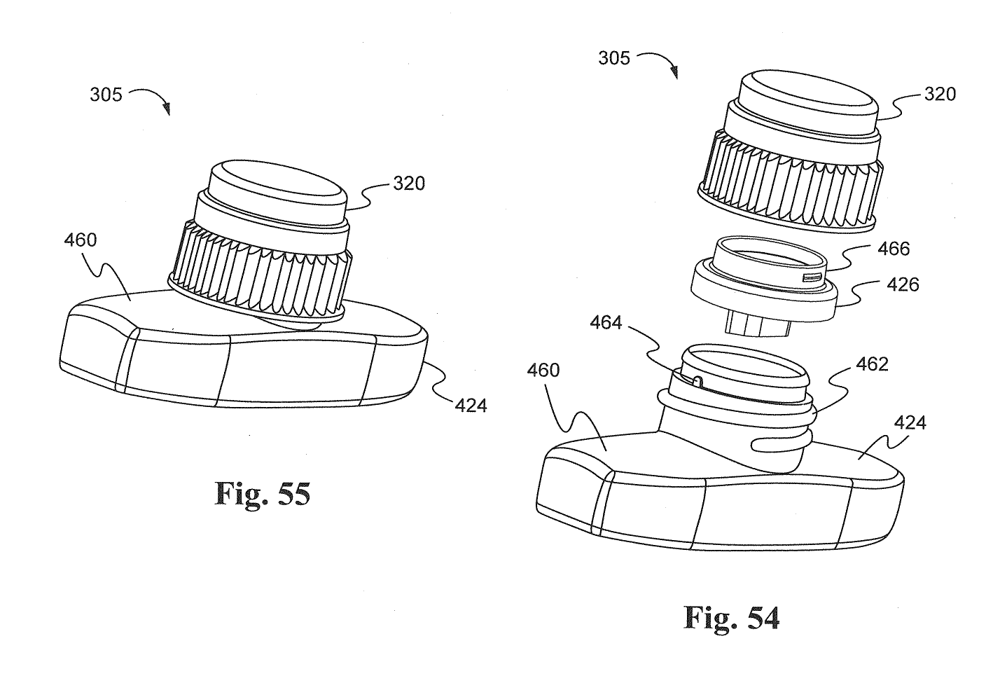

In general, the container assembly 305 can be positioned within the base 304 and can include a bottle or container 316, as well as a plug 318. As will be additionally discussed below, in some other embodiments, the container assembly 305 can also include a bottle cap 320 (See FIGS. 54 and 55 below). The container assembly 305 in turn can receive the shroud assembly 306, which in at least some embodiments, can include a pair of wick holders (also referred to herein as inner shroud members or wick retainers) 322 for holding the wick 308 in position and a shroud (also referred to herein as an outer shroud member) 324 for securing the wick holders 322 in position with respect to the container assembly. Each of the pair of panels 310 can be disposed within the base 304, in a manner described below, to complete the assembly of the wick diffuser 300.

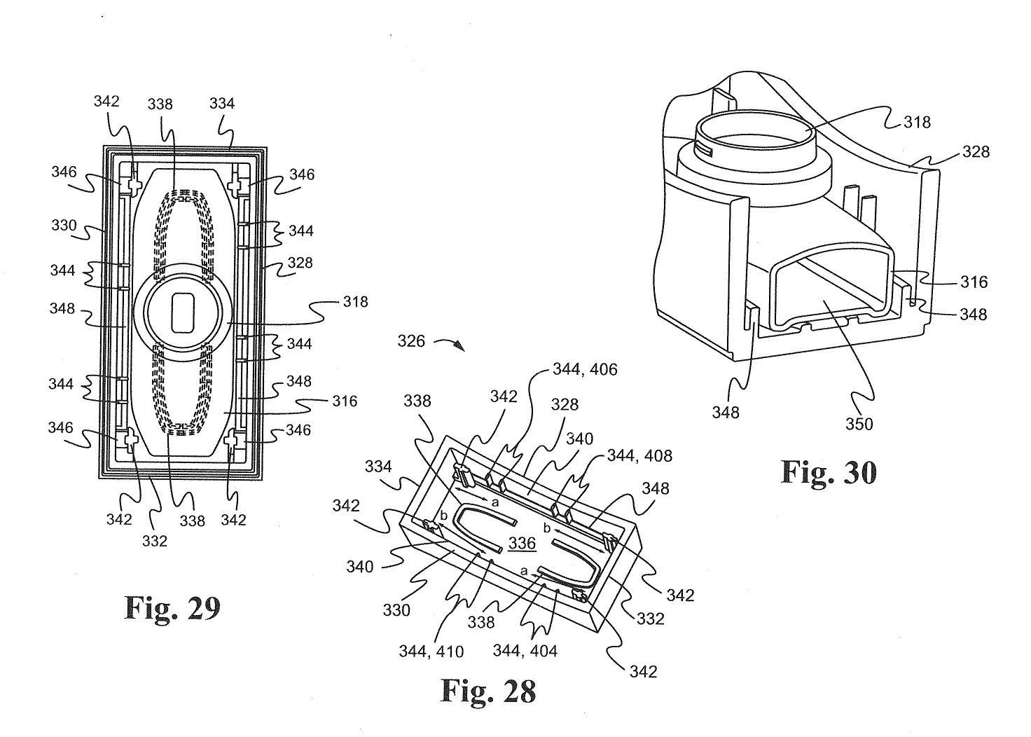

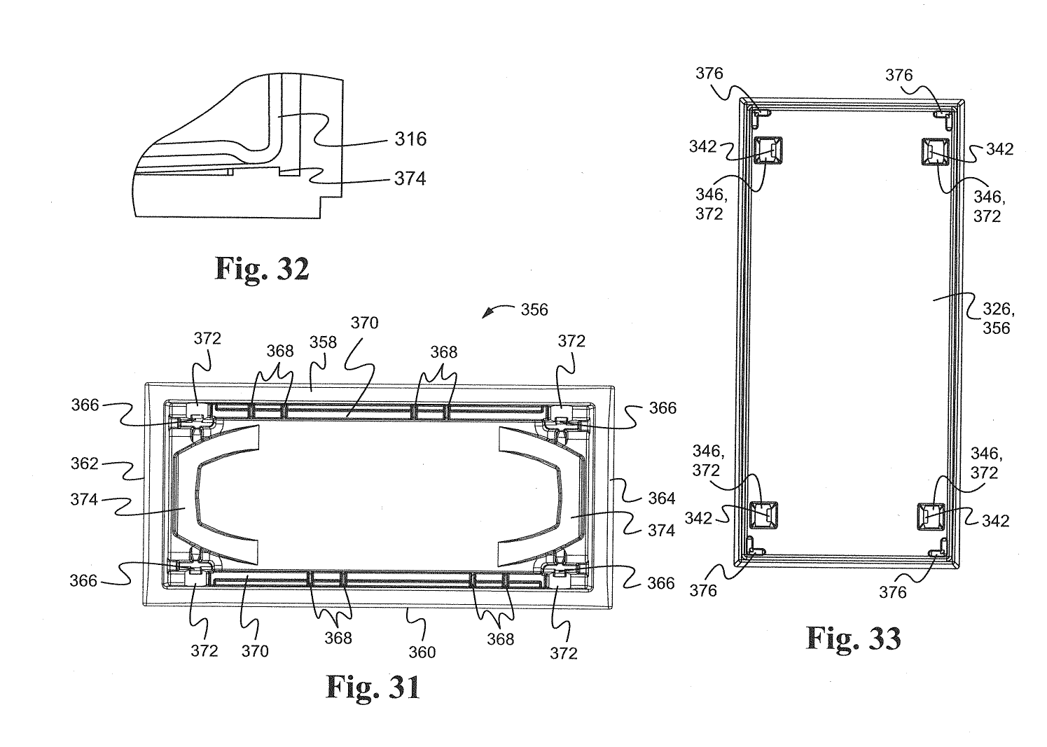

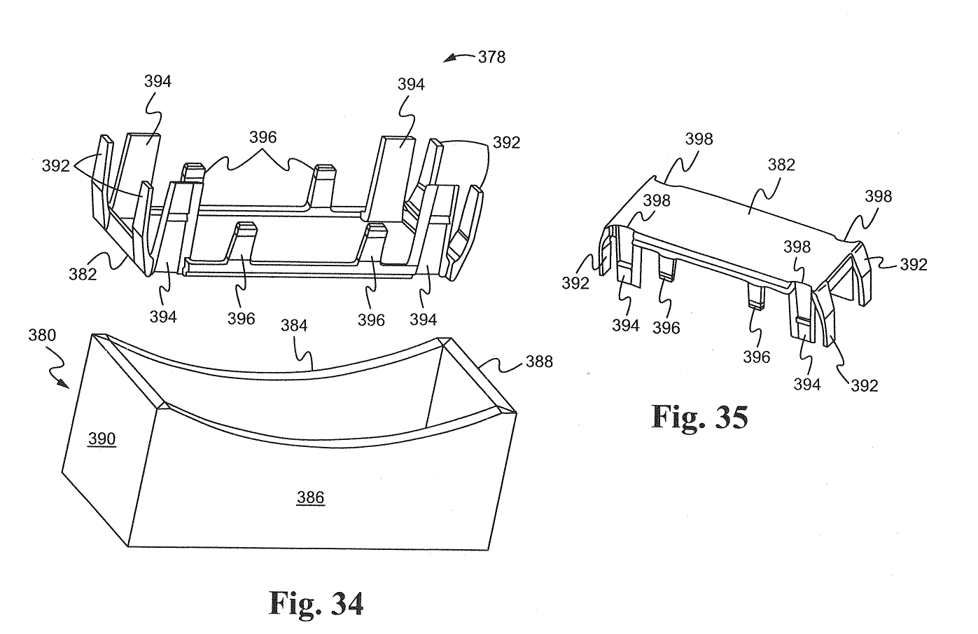

Turning now to FIGS. 28-35, various embodiments of the base 304 are described in greater detail. In particular, FIGS. 28-30 show a first embodiment of the base 304, while FIGS. 31 and 32 show a second embodiment of the base. FIG. 33 shows a bottom view of the first and the second embodiments of the base 304, while FIGS. 34-35 show a third embodiment of the base 304.

Referring now to FIGS. 28-30 and to FIG. 28 in particular, a perspective view of the first embodiment of the base 304, namely, a base 326 is shown, in accordance with at least some embodiments of the present disclosure. As shown, the base 326 can include elongated first and second arcuate wall members 328 and 330, respectively, which mate with respective shorter third and fourth non-arcuate wall members 332 and 334 to form an enclosed structure. In at least some embodiments, each of the third and fourth non-arcuate wall members 332 and 334, respectively, can be designed to angle slightly outwardly to receive the pair of panels 310 and be arcuate as well or can possibly assume other configurations suitable for receiving the panels, as well as the container assembly 305 within the base 326.

Similarly, the first and the second wall members 328 and 330, respectively, need not be arcuate in other embodiments and, can assume other configurations suitable for fittingly receiving the pair of panels 310 and the container assembly 305 and can angle slightly outwardly as well. Furthermore, the wall members 328-334 need not always be configured to form a rectangular structure, as shown. In some embodiments, the geometry of the base 326 can vary along with the geometry of the container assembly 305.

The base 326 can further include a floor member 336 on a top surface of which are formed a pair of ribs 338. The ribs 338 are described in greater detail below. Also formed along an inner wall 340 of the first and second wall members 328 and 330, respectively, are spring fingers 342, as well as keying ribs 344. In at least some embodiments, two pairs of spring fingers 342, each pair disposed along an inner corner of the base 326, and four pairs of keying ribs 344, two pairs being disposed along the inner wall 340 of each of the first and second wall members 328 and 330, respectively, can be present.

With respect to the spring fingers 342 in particular, they provide at least moderate retention of the pair of panels 310 within the base 326 while also allowing a user to remove those panels for access to the refill (container assembly 305 plus wick 308 plus shroud assembly 306). The spring members 342 serve to receive the pair of panels 310 in a manner similar to that described above with respect to FIGS. 1-24. In at least some embodiments, the spring fingers 342 can be formed by holes 346 (See FIGS. 29 and 33), which can extend from the top surface of the floor 336 to a bottom surface thereof to form the aforementioned spring fingers. In at least some other embodiments, the spring fingers 342 can be formed by way of other features.

While the spring fingers 342 can serve to retain the pair of panels 310, the keying ribs 344 can prevent the user from inserting the panels 310 incorrectly, as described in greater detail below. Together, the spring fingers 342 and the keying ribs 344 ensure that the panels 310 are secured properly and correctly within the base 304 (such as the base 326). Furthermore, in order to prevent liquid (or fragrance) from escaping through the holes 346 in the base 326 in any unintended leakage situation of the container assembly 305, a well 348 can be formed along the inner wall 340 of the first and second wall members 328 and 330, respectively (only one of the wells is visible in FIG. 28).

The well 348 can extend vertically upward from the top surface of the floor member 336 such that a bottom surface of the pair of panels 310 bottom out on a top surface of the well. The keying ribs 344 can also extend upwardly from the top surface of the well 348 to receive the pair of panels 310, as described below. In at least some embodiments, the well 348 can be designed to be a six (6) millimeter well. In other embodiments, the configuration and dimensions of the well 348 can vary.

With respect to the ribs 338 in particular, each of the pair of ribs is U-shaped or substantially U-shaped to receive and stabilize the container assembly 305 and, particularly, the bottle 316 of the container assembly. In particular, and as described in greater detail below, in at least some embodiments, a floor 350 (See FIG. 30) of the bottle 316 can be tapered to direct the liquid therewithin toward a center portion of the bottle for efficient dissemination of the fragrance. Such a tapered configuration of the floor 350 can render the bottle 316 unstable causing undesired rocking of the bottle when the wick diffuser 300 is moved.

In order to prevent such nuisance rocking of the bottle 316, the ribs 338 can be provided to support and stabilize the bottle during movement of the wick diffuser 300. Particularly, the ribs 338 can be designed to mimic the taper of the floor 350 of the bottle 316, tapering from the end closer to the end walls 332 and 334 towards the center of the base 326. The perimeter of the bottle 316 can extend beyond the edges of the ribs 338, as shown in FIGS. 29 and 30.

Turning now to FIGS. 31 and 32, a second embodiment of a base 356 is shown, in accordance with at least some embodiments of the present disclosure. Similar to the base 326, as shown in FIG. 31, the base 356 can include long arcuate wall members 358 and 360, which mate with slightly outwardly angled non-arcuate shorter wall members 362 and 364 to form an enclosed structure. Further, similar to the base 326, the base 356 can also include two pairs of spring fingers 366 and four pairs of keying ribs 368 to secure the pair of panels 310, a well 370 for containing any spillage, holes 372 to foi in the spring fingers, as well as a pair of ribs 374 to hold the bottle 316 of the container assembly 305 in a stable fashion.

Also similar to ribs 338, the ribs 374 can be U-shaped or substantially U-shaped (or even horse-shoe shaped) and mimic the taper of the floor 350 of the bottle 316 towards the center portion of the bottle for providing stability thereto. In contrast to the ribs 338, however, the ribs 374 are formed further towards the shorter wall members 362 and 364 and, are wider than the ribs 338 for supporting the perimeter of the bottle 316. Thus, in contrasts to the ribs 338 in which the perimeter of the bottle 316 extends beyond the boundaries of the ribs, the ribs 374 are designed such that the perimeter of the bottle rests on the surface of the ribs.

Turning now to FIG. 33, a bottom view of the bases 326 and 356 is shown, in accordance with at least some embodiments of the present disclosure. In addition to the holes 346 and 372 of the bases 326 and 356, respectively, two pairs (four) of legs (or base feet) 376 can be formed on four corners of the bottom surface of the bases. In at least some embodiments, each of the two pairs of legs 376 can be L-shaped or substantially L-shaped, and can prevent a user from observing any lack of flatness in the surface on which the wick diffuser 300 is placed by raising the base 326, 356 up to contact that surface only on the four legs. In at least some other embodiments, the shape of the legs can vary.

Turning now to FIGS. 34 and 35, a third embodiment of a base 378 is shown, in accordance with at least some embodiments of the present disclosure. As shown, the base 378 can include a base structure 380 and a chassis 382. In at least some embodiments, similar to the bases 326 and 356, the base structure 380 can be formed as an enclosed structure with arcuate wall members 384 and 386, which mate with non-arcuate wall members 388 and 390. Also similar to the bases 326 and 356, the non-arcuate wall members 388 and 390 can be angled slightly outwardly, although this need not always be the case. The base structure 380 can be designed to receive therein the chassis 382 on which are formed two pairs of resilient cam members 392 that snap/friction fit within the base structure upon being placed therein.

In at least some embodiments, the base structure 380 (and/or the chassis 382) can be constructed of wood (such as bamboo) although in other embodiments, the base structure (and/or the chassis) can be constructed of plastic or other suitable material. Relatedly, the bases 326 and 356 can each be constructed of wood, plastic or other suitable material.

As further shown, the chassis 382 can be dimensioned to be completely enclosed and surrounded by the base structure 380 when positioned therewithin. The chassis 382 can further include four spring fingers 394 and keying ribs 396 for securing the panels 310. Although only two pairs of keying ribs 396 have been shown in the present embodiment, it will be understood that in at least some other embodiments, four pair of keying ribs, as shown with respect to the bases 326 and 356, can be present. It will similarly also be understood that the bases 326 and 256 can be designed with only two pairs of keying ribs 344 and 374, respectively, with a pair of keying rib along each side of the inner wall 340.

Furthermore, in contrast to the spring fingers 342 and 366, the spring fingers 394 are not formed with holes 346 and 372, respectively, on the respective bases 326 and 356. Rather, the spring fingers 394 can be formed with arcuate cut-outs 398 on a bottom surface of the chassis 382, as shown in FIG. 35, but nonetheless serve the same purpose as the spring fingers 342 and 366. Also, even though the chassis 382 is not shown to include any ribs on the floor thereof, it will be understood that in at least some embodiments, such ribs can nevertheless be present for stabilizing the container assembly 305.

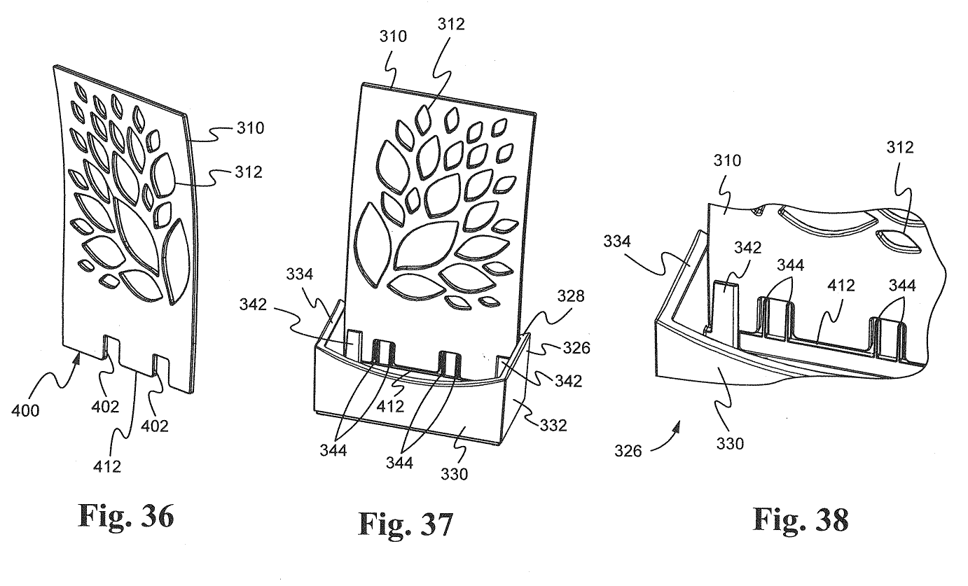

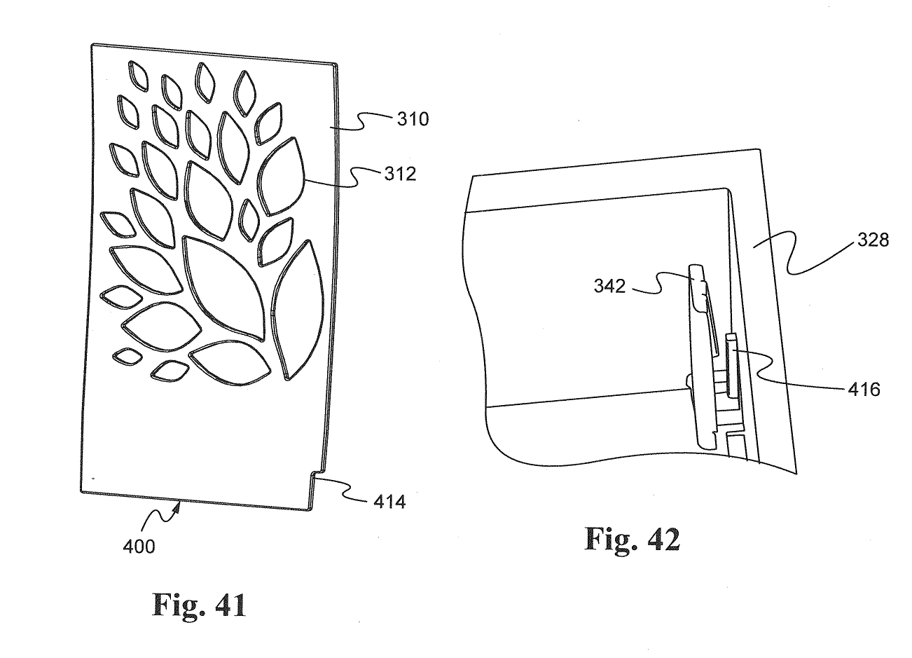

Referring now to FIGS. 36-46, two embodiments for securing/assembling or keying-in the pair of panels 310 within the base 304 are shown, in accordance with at least some embodiments of the present disclosure. In particular, FIGS. 36-40 show a first embodiment of keying-in the pair of panels 310, while FIGS. 41-46 show a second embodiment thereof. For conciseness of expression, the keying-in of the panels 310 of FIGS. 36-46 below is described with respect to the keying ribs 344 and other components of the base 326. However, it will be understood that a similar keying-in procedure would apply to the keying ribs 368 and 396 of the bases 356 and 378, respectively.

Referring now to FIGS. 36-40 and to FIG. 36 in particular, one of the pair of panels 310 is shown having the pattern 312. A bottom portion 400 of each of the pair of panels 310 can be designed with a pair of non-symmetrical slots/cut-outs 402 that can correspond in position to align with the keying ribs 344 within the base 326. Assembling each of the pair of panels 310 correctly within the base 326 assists in the proper operation of the wick diffuser 300. For example, if the pair of panels 310 are installed backward (e.g., with the arc of each of the pair of panels facing inwards), the pair of panels can potentially make contact with the wick 308 and provide a path for fragrance migration.

Thus, to help ensure that each of the pair of panels 310 is assembled correctly within the base 326, the keying ribs 344 can be designed to be asymmetrical or be at unequal distances from the wall members 332 and 334, as shown more clearly in FIGS. 28 and 31 above. More particularly and referring back to FIG. 28, a first and second pair 404 and 406, respectively, of the keying ribs 344 can be constructed such that they are positioned at a distance “a” from the respective wall members 332 and 334, while a third pair 408 and a fourth pair 410 can be positioned at a distance “b” from those wall members. The distance “a” and “b” can be different from each other to ensure that the positioning of the keying ribs 344 is asymmetrical.

Referring back to FIG. 36, corresponding to the distances “a” and “b,” the slots 402 on the bottom portion 400 each of the pair of panels 310 can also be formed at distances “a” and “b.” Furthermore, the slots 402 can be dimensioned (e.g., length and width) to securely fit around one of the pairs of the keying ribs 344. Accordingly, each of the pair of panels 310 can be assembled by aligning the slots 402 with two pairs of respective keying ribs 344 (such as the second pair and third pair 404 and 408, respectively) and sliding the panels about those keying ribs. By virtue of providing the keying ribs 344 and, particularly providing the keying ribs in an asymmetrical fashion as described above, incorrect placement of each of the pair of panels 310 can be avoided. If the pair of panels 310 are assembled correctly, the arcuate surface of those panels will face outwardly (e.g., curving away from the base 326) as shown in FIGS. 37 and 38.

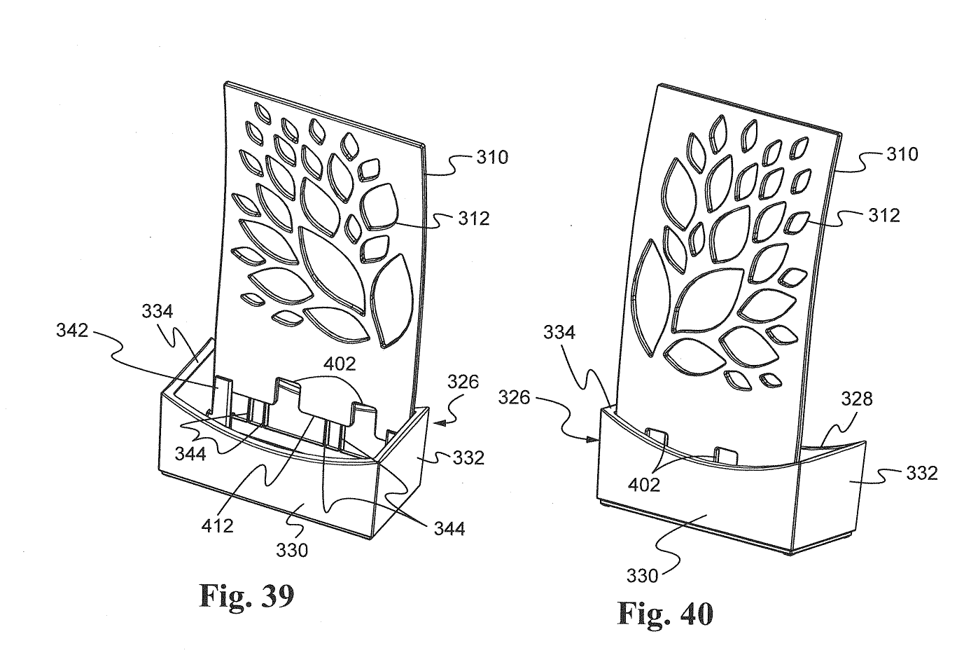

On the other hand, and as shown in FIGS. 39 and 40, if the pair of panels 310 are installed (or otherwise assembled) backwards (i.e., with the arcuate surface facing inwards or the curvature of the panels facing towards the base 326), the slots 402 on the bottom portion 400 of those panels will not align with the keying ribs 344, thereby causing the bottom portion of those panels to bottom out on the top surface of the keying ribs. Furthermore, and as shown more clearly in FIG. 40, if any of the pair of panels 310 is installed incorrectly, the slots 402 will be visible from the top of the base 326 and those panels will be tippy. Thus, the keying ribs 344 ensure proper installation of the pair of panels 310 and additionally serve to provide cues to the user if the panels are installed incorrectly.

Furthermore, it will be understood that although the above description is provided with two pairs of the keying ribs 344 on either side of the arcuate wall members 328 and 330, in at least some other embodiments, only two pairs of the keying ribs, one pair on either side of those wall members can be employed. Correspondingly, each of the pairs of panels 310 may also only have one of the slots 402 on the bottom portion 400 thereof. In such embodiments, the keying ribs 344 can be offset from one another and correspondingly, the slots 402 on the pair of panels 310 can be offset to align with the offset keying ribs. If the pair of panels 310 are assembled correctly, the slots 402 will be seated about the keying ribs, while if any of those panels are not assembled correctly, the bottom portion 400 of the panels will bottom out on the top surface of those keying ribs.

Additionally, in at least some embodiments, a middle portion 412 of the bottom portion 400 of the pair of panels 310 can be shortened in order to clear any burr left behind by the construction (such as, injection molding) process. Also, as described above, the non-arcuate wall members 332 and 334 can angle slightly outwardly. Such an angling provides for the required draft during the construction process. Each of the pair of panels 310 can, in at least some embodiments, taper inwardly at the same location to ensure a snug fit to the base 326.

Turning now to FIGS. 41-46, a second embodiment of assembling the pair of panels 310 within the base 326 is shown, in accordance with at least some other embodiments of the present disclosure. Referring in particular to FIG. 41, one of the pair of panels 310 having the pattern 312 is shown. Instead of the slots 402, each of the pair of panels 310 can include an L-shaped notch 414 formed on a corner of the bottom portion 400 of each of the panels. The notch 414 can be designed to rest over and about a corresponding a keying rib 416, shown in FIG. 42, formed at a corner along the arcuate wall members 328 and 330. The keying rib 416 is different from the keying ribs 344 in that only a pair of the keying ribs 416 (one on each side of the wall member 328 and 330) is present in contrast to the four pair (or two pairs) of keying ribs 344.

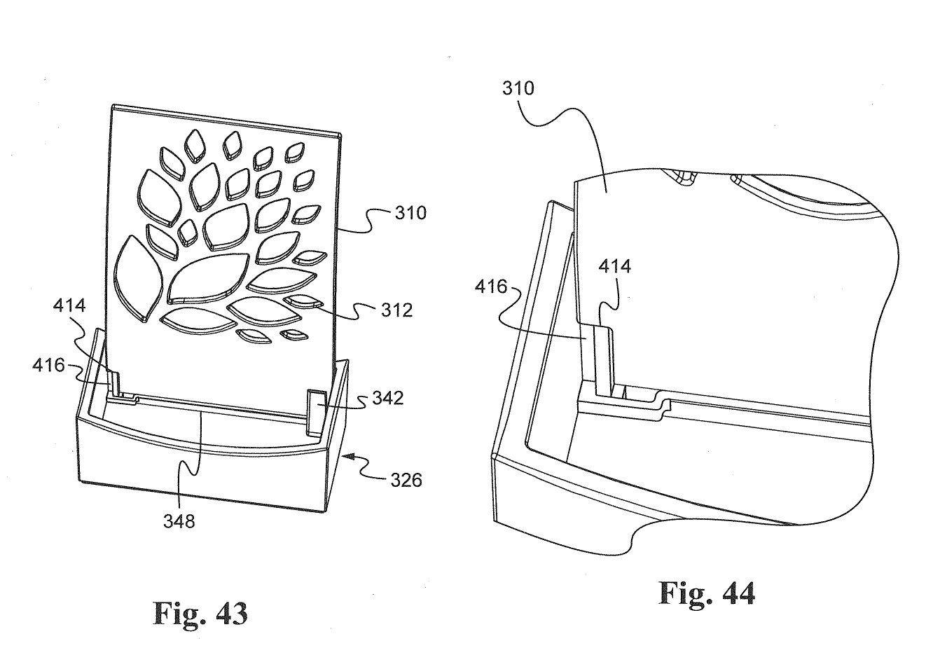

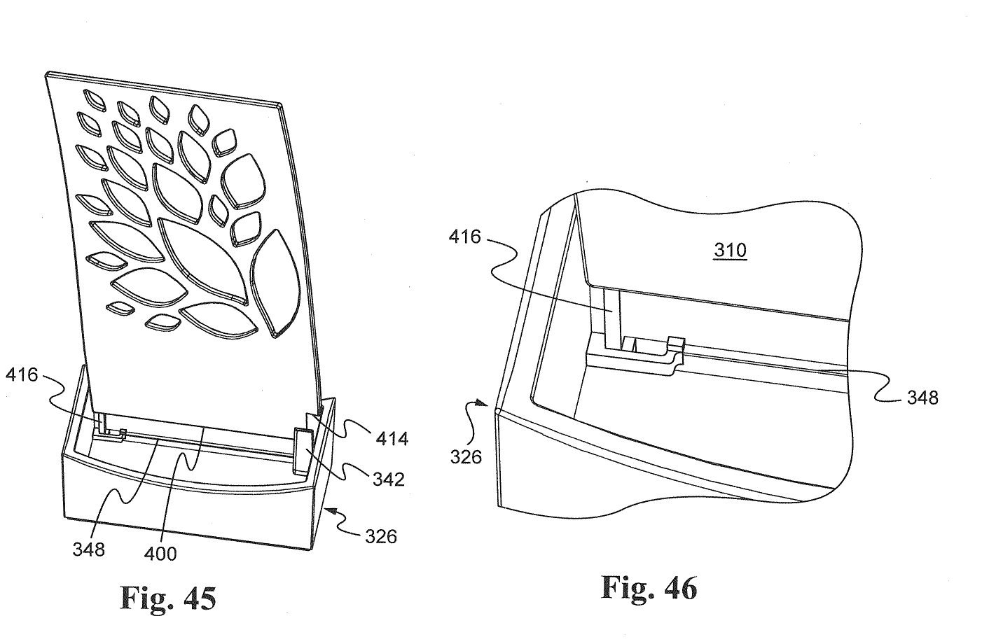

If the pair of panels 310 are assembled correctly, the notch 414 will rest over and about the keying rib 416, as shown in FIGS. 43 and 44 with the bottom portion 400 of those panels resting on the top surface of the well 348. On the other hand, if any of the pair of panels 310 is assembled backwards, then as shown in FIGS. 45 and 46, the bottom portion 400 of the panel will bottom out on the top surface of the keying rib 46. Additionally, if assembled incorrectly, the pair of panels 310 will be unbalanced and wobbly.

It will be understood that in at least some embodiments, either the keying rib 416 or the keying ribs 344 can be present to facilitate assembly of the pair of panels 310. However, in at least some other embodiments, a combination of the keying rib 416 and the keying ribs 344 can be present as well to securely assemble the pair of panels 310 within the base 326.

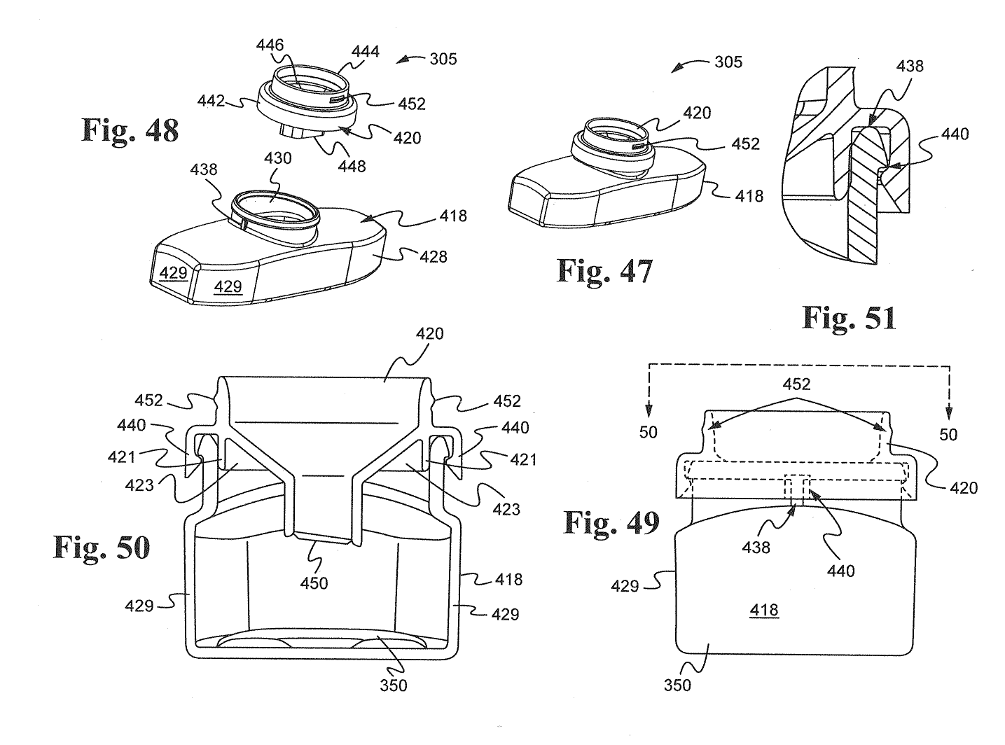

Referring now to FIGS. 47-55, two embodiments of the container assembly 305 are described in greater detail below. In particular, FIGS. 47-53c show a first embodiment of the container assembly 305, which can include a non-threaded bottle 418 and a plug 420, while FIGS. 54 and 55 show a second embodiment of the container assembly, which can include a threaded bottle 424, a plug 426 and a bottle cap 320. It will be understood that although only two embodiments of the container assembly 305 are described below, various other configurations suitable for use with the base 304 can be employed in other embodiments.

Referring now particularly to FIGS. 47-53c and, more particularly, to FIGS. 47 and 48, assembled and exploded views, respectively, of the container assembly 305 are shown, in accordance with a first embodiment of the present disclosure. As shown, the container assembly 305 can include the non-threaded bottle 418 and the plug 420. The bottle 418 can further include a body portion 428 defining a cavity therewithin for storing the VOC liquid and dimensioned to be completely contained within the base 304, as well as a neck portion 430 extending upwardly and transversely from the body portion to receive the plug 420. In at least some embodiments, the bottle 418 and the plug 420 can be designed such that at least a portion of the bottle 418 projects out from the top of the base 304.

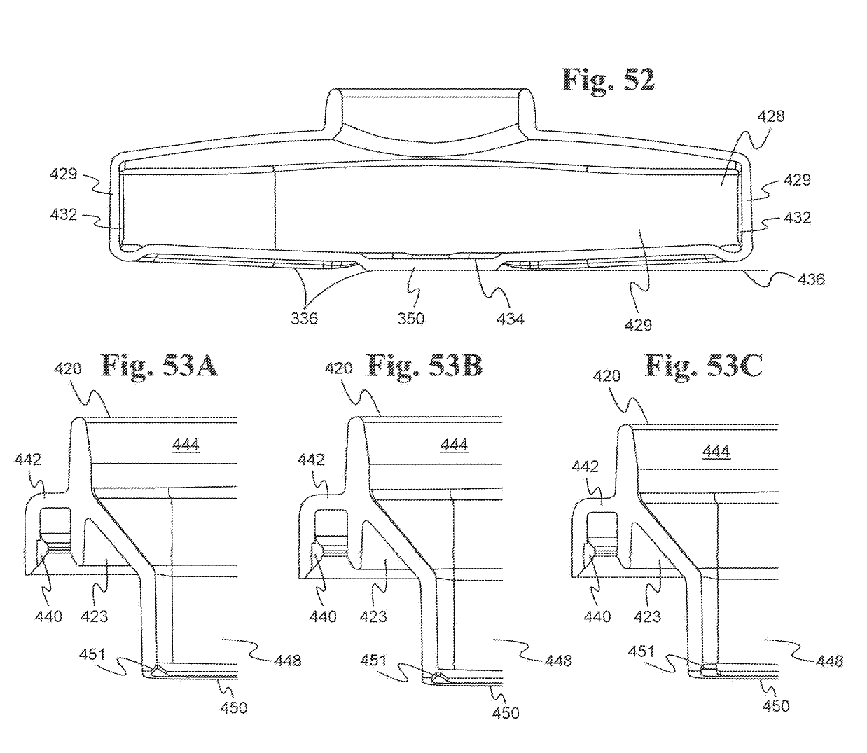

With respect to the bottle 418 in particular, and as shown in FIG. 52, the body portion 428 of the bottle can include, among other side walls 429, the floor 350, which as described above can be tapered in at least some embodiments to facilitate the migration of the liquid stored within the bottle toward the wick 308 for an efficient dissemination of fragrance therethrough. The floor 350 of the bottle 418 can taper from end portions 432 toward a center portion 434 of the bottle. As also seen from FIG. 52, the floor 350 can be designed such that, when disposed within the base 304, the center portion 434 can rest on the top surface of the floor member 336 of the base, while the end portions 432 can be slightly raised and form an angle with the center portion, as well as the floor member, as shown by line 436. Although this tapered configuration of the floor 350 permits efficient dispersal of fragrance, as described above, it can also render the bottle 418 unstable during movement of the wick diffuser 300. Accordingly, the end portions 432 of the floor 350 can be supported on the aforementioned ribs 338 and 374 fotnied on the top surface of the floor member 336.

Turning back to FIGS. 47 and 48, the neck portion 430 of the bottle 418 can incorporate lugs 438. Although only one of the lugs 438 is visible in FIGS. 47 and 48, a pair of lugs on diametrically opposite ends of the neck portion 430 can be present in at least some embodiments, as shown in FIG. 50. In some other embodiments, only a single lug or possibly more than two lugs can be present as well. The lugs 438, in particular, are anti-rotational lugs, each of which are designed to be in mating alignment with corresponding anti-rotational lug bypass members 440 on the plug 420, as seen more clearly in FIGS. 49 and 50.

With respect to the plug 420, as seen from FIG. 48, it can include an outer rim 442 dimensioned to securely fit around the neck portion 430 of the bottle 418 and a smaller inner rim 444 defining an opening 446 and leading to a stem 448 for receiving the shroud assembly 306 and the wick 308, as described further below. The plug 420 can be secured around the neck portion 430 of the bottle 418 by positioning the outer rim 442 of the plug around the neck portion of the bottle and rotating the plug until the anti-rotation lug bypass members 440 formed on an inner surface of the outer rim mate and lock with the lugs 438, as seen more clearly in FIG. 51. An assembled view of the bottle 418 and the plug 420 is shown in FIG. 47.

By virtue of providing the lugs 438 and locking them with the anti-rotational lug bypass members 440, the user can be prevented from accidently (or intentionally) removing the plug 420 from the bottle 418 and accessing the liquid within. Further, by securing the plug 420 to the bottle 418 in the manner described above, a wiping seal 421 is provided to seal the plug to the bottle during shipping, thereby ensuring that the bottle can withstand any low ambient pressures that are commonly expected during shipping of the product. The wiping seal 421 in particular is formed by a sealing rib 423 of the plug 420 that contacts with an inner surface of the neck portion 430 of the bottle 418. The sealing rib 423 can be designed such that it is only connected to the plug 420 at an upper portion thereof, thereby capable of flexing and conforming to the shape of the neck portion 430. Such a flexibly constructed seal can be particularly important when during construction of the neck portion 430 of the bottle 418, any level of ovality is observed in the neck portion, which can prevent a proper sealing between the plug 420 and the neck portion of the bottle. However, by virtue of providing the flexible sealing rib 423, the wiping seal 421 can better conform to the shape of the neck portion 430.

In addition to the anti-rotational lug bypass members 440, the plug 420 can also include a pair of retention members 452, which as shown in FIGS. 47-50 and described below, can be employed for securing the shroud assembly 306 to the bottle 418 via the plug 420. Furthermore, in order to prevent activating the diffuser prior to installing the shroud assembly 306 within the container assembly 305, a bottom portion of the stem 448 of the plug 420 can be equipped with a puncturable membrane 450, as seen more clearly from FIGS. 53a-c.

In order to facilitate puncturing the membrane 450, the membrane can be provided at a compound angle, as shown in FIG. 50, thereby ensuring a single point of entry and reducing the insertion force required for the wick 308 to puncture the membrane. In at least some embodiments, the membrane 450 can be manufactured by the “coining” process, described above, to provide a scored line/coined seal or a line of relative weakness around the perimeter between the membrane 450 and the plug 420. Accordingly, when the user downwardly presses the shroud assembly 306 against the sealing membrane 450, the membrane or “coin” may be ruptured thus causing the remainder of the membrane to downwardly depend from the plug in a hanging fashion. The membrane 450 can be dimensioned so as to not engage the floor 350 of the bottle 418, 424 even after being ruptured to prevent any interference with the wick 308.

Furthermore, and as shown in FIGS. 53a-c, the membrane 450 can be provided in a variety of manners such that an activation force required to puncture the membrane is optimized. For example, as shown in FIG. 53a, the coined seal of the membrane 450 can be triangle-shaped where the seal is shaped like a triangle with a pointed tip 451, or as shown in FIG. 53b, it can be round-shaped where the seal is triangle-shaped but with a rounded tip 453. As also shown in FIG. 53c, the coined seal can be rectangular-shaped where a seal 455 is flat and shaped like a rectangle.

In addition, the stem 448 and, hence the membrane 450, of the plug 420 can be designed such that a relative gap between the shroud assembly 306 and the plug is reduced when activation of the refill is desired. By doing so, a pinch hazard where the user can pinch his/her finger/flesh between the gap can be avoided. To further avoid the pinch hazard, the coined seal can be designed to hinge on the longer end of the membrane 450 with respect to the floor 350 of the bottle 418, 424, which in turn can reduce the gap between the shroud assembly 306 and the plug 420 prior to puncturing the membrane.

Turning now to FIGS. 54 and 55, a second embodiment of the container assembly 305 is shown, in accordance with at least some teachings of the present disclosure. The second embodiment of the container assembly 305 is similar to the first embodiment thereof and, for conciseness of expression, only the differences between the two embodiments have been set forth below. In particular, the second embodiment can include the bottle 424 and the plug 426. The bottle 424, similar to the bottle 418, can include a body portion 460 dimensioned to fit within the base 304 and having a tapered floor (such as the floor 350), as well as a neck portion 462 for receiving the plug 426. However, in contrast to the first embodiment, the neck portion 462 of the bottle 424 can be threaded and designed to receive the bottle cap 320, which in turn can include threads on an inner surface thereof for mating with the outer threads on the neck portion.

The bottle cap 320 can typically be assembled (e.g., screwed) over the neck portion 462 of the bottle 424 during shipping of the wick diffuser 300, as well as up to the point when the user is ready to activate the wick diffuser. The bottle cap 320 can be assembled over the neck portion 462 by placing the bottle cap over the neck portion and rotating the bottle cap until the bottle cap is tightly secured over the neck portion. To remove the bottle cap 320 (e.g., when activating the product), the user can counter-rotate and lift the bottle cap from the neck portion 462 of the bottle 424.

The bottle 424 can further include anti-rotational lugs 464 designed to mate with lugs (not visible) on an inner surface of the plug 426, which can be similar in design and construction to the plug 420 described above. The plug 426 can additionally include retention members 466 for securing the shroud assembly 306, as described further below, as well as a rupturable membrane (not visible) for promoting contact of the wick 308 with the liquid within the bottle 424.

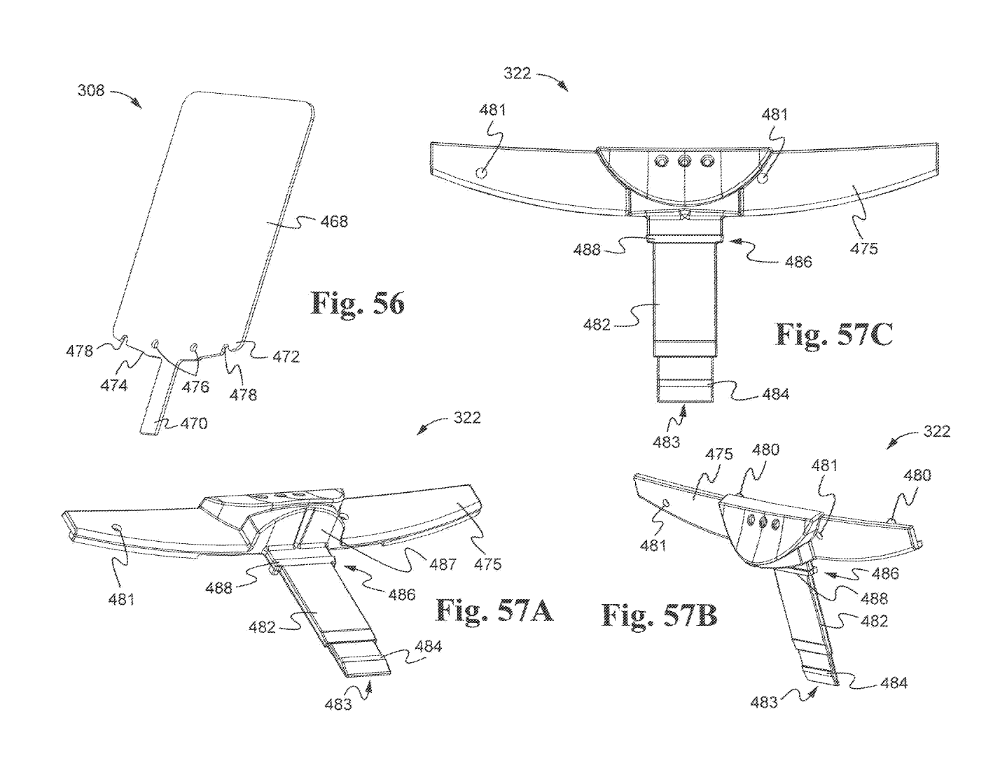

Referring now to FIG. 56, a perspective view of the wick 308 is shown, in accordance with at least some embodiments of the present disclosure. The wick 308, similar to the wick described with respect to the wick in FIGS. 1-24, can have a planar surface and capable of being received within the shroud assembly 306, in a manner described below. In particular, in at least some embodiments, the wick 308 can include a body portion 468 and a stem portion 470. Fillets 472 can be provided at the transition from a bottom edge 474 to the stem 470 to increase the strength of the stem, at least a portion of which can penetrate the aforementioned rupturable membrane 450 when use is desired.

Furthermore, in order to securely hold the wick 308 in position during use of the wick diffuser 300, the wick 308, as described above, can be secured within the shroud assembly 306. To facilitate the foregoing, the wick 308 can have formed on the bottom edge 474 thereof, a plurality of holes and notches, such as, a pair of inner holes 476, as well as a pair of outer notches 478, which can mate and lock with corresponding features on the shroud assembly 306 and, in particular, the pair of wick holders 322, described below.

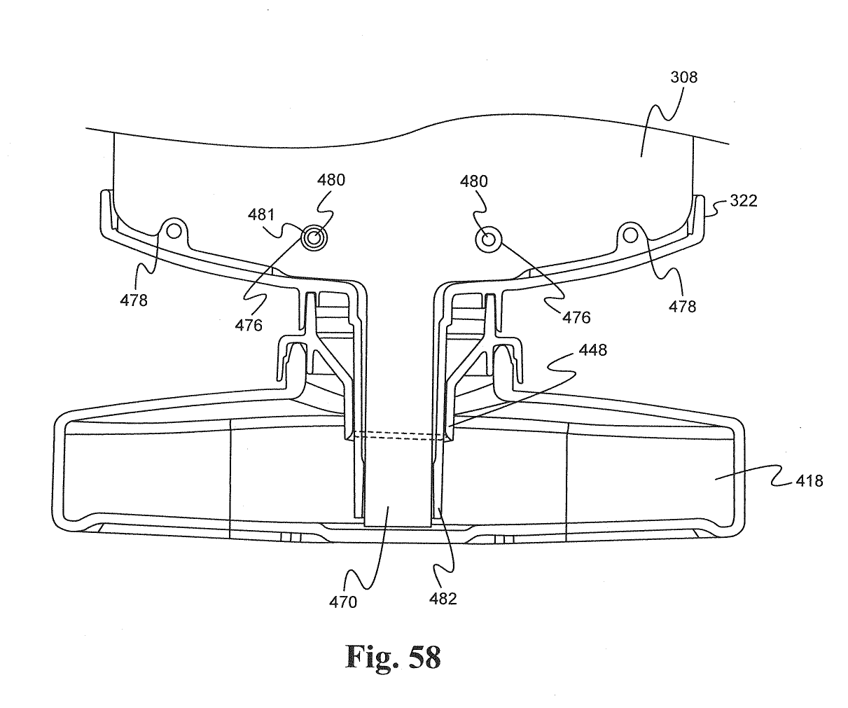

Turning now to FIGS. 57a-57c, various views of the wick holders 322 are shown, in accordance with at least some embodiments of the present disclosure. In particular, FIGS. 57a and 57b show perspective views of the wick holders 322, while FIG. 57c shows a front view thereof. The wick holders 322 can be designed such that upon assembly, the wick 308 is sandwiched in a secure manner between the pair of the wick holders. To facilitate such an assembly, a lateral member 475 of each of the pair of wick holders 322 can be equipped with a pair of posts 480, one of which can correspond in location to one of the inner holes 476 on the wick 308, while the other of the posts can correspond in location to one of the outer notches 478, as well as a pair of holes 481, which serve to receive the posts of the other of the pair of wick holders. The wick 308 can be secured to the wick holders 322 by aligning its holes 476 and notches 478 with the posts 480 on the wick holders and snapping the wick to the wick holders. A view of the wick 308 assembled to one of the wick holders 322 is shown in FIG. 58, which is a partial front cross-sectional view showing the bottle 418 and the shroud assembly 306 assembled with respect to the bottle.

Each of the wick holders 322 can further include a stem 482 extending transversely from the lateral member 475. In at least some embodiments, and as shown in the assembled view of FIG. 58, the stem 482 can be dimensioned in width to completely contain the width of the stem 470 of the wick 308, while being shorter in length than the stem of the wick to facilitate contact of the wick with the VOC upon assembly to the container assembly 305 during usage of the wick diffuser 300. As also shown in FIG. 58, upon assembly, the length of the stems 482 and 470 of the wick holders 322 and wick 308, respectively, can be typically longer than the length of the stem 448 of the plug 420 (and the plug 426), but does not contact the floor 350 of the bottle 418 (or the bottle 424).

Turning back to FIG. 57a-c, a bottom portion 483 of the stem 482 of each of the wick holders 322 can further include on an outer surface (e.g., the side opposing the side securing the wick 308) thereof, a catch-type interface 484, which can be designed to provide a unidirectional attachment of the wick holders to the outer shroud member 324 to prevent disassembly of the wick holders once they are installed to the outer shroud member. Also provided on a top portion 486 of the outer surface of each of the wick holders 322, are additional retention members 488, which provide a second point of connection of the wick holders to the outer shroud member 324, thereby minimizing (or possibly completely preventing) any relative movement therebetween. For added fragrance dispersal and for avoiding VOC splashing during activation and usage of the wick diffuser 300, an aperture, hole or cut out in a bottom surface 487 of the wick holders 322 may be designed in some embodiments to provide a path for any VOC that may splash during activation (or usage) of the wick diffuser. Other or additional features to prevent splashing may be added in other embodiments.

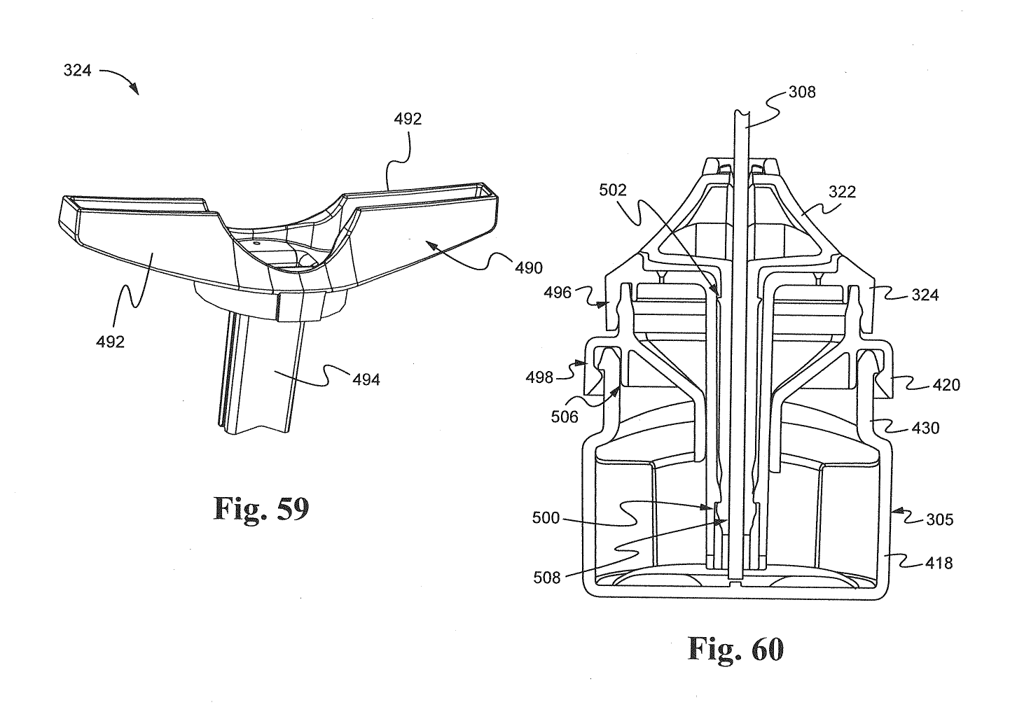

Referring now to FIG. 59, a perspective view of the outer shroud member 324 is shown, in accordance with at least some embodiments of the present disclosure. As shown, the outer shroud member 324 can include a lateral body section 490 having first and second members 492 defining a space therebetween for securely receiving the wick 308 and the pair of wick holders 322. The outer shroud member 324 can further include a stem 494, also defining a space for receiving the stems 470 and 482 of the wick 308 and the wick holders 322, respectively. The wick holders 322 (to which the wick 308 has already been attached) can be secured to the outer shroud member 324 by inserting the stem 482 of the wick holders within the stem 494 until the catch type interface 484 and the retention members 488 of the wick holders contact and lock with corresponding features formed within the stem of the outer shroud member 324. Various gussets, grooves and other features may be provided on the outer shroud member 324 to limit part deformation during ejection from the molding tool during construction. Furthermore, the outer shroud member 324 may be designed with one or more spill/drip proof features to contain any dripping and/or migrating VOC from the wick 308 and to direct that VOC back into the container assembly 305. For example, in at least some embodiments, the outer shroud member 324 may be designed with a cup-like or basin-like feature (such as that shown in FIG. 59) that may capture the excess VOC from the wick 308 and transfer that excess VOC back into the container assembly 305 through the stem 494. In other embodiments, other or additional types of spill/drip proof features may be added to the wick holders 322 and the outer shroud member 324.

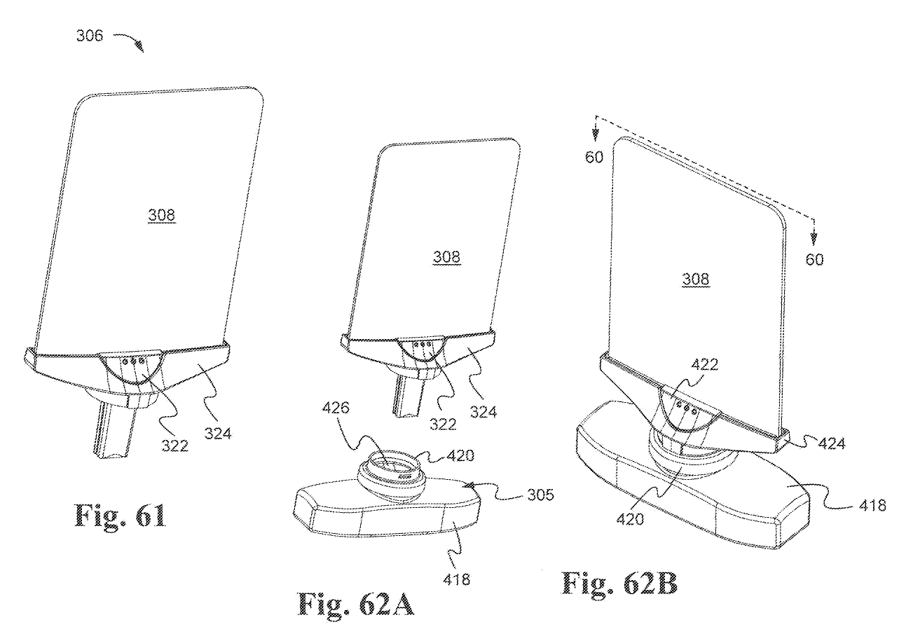

An assembled view of the shroud assembly 306 is shown in FIG. 61. As shown in FIG. 61, the wick 308 may be inserted between the pair of wick holders 322 (only one of which is visible) and then the wick holders plus the wick may be inserted within the outer shroud member 324, thereby forming the shroud assembly 306. It will be understood that the aforementioned assembly of the wick 308, the wick holders 322 and the outer shroud member 324 can be performed by the user when use of the wick diffuser 300 is desired or alternatively, one or more of the aforementioned assemblies can occur during manufacture of the wick diffuser.

The shroud assembly 306 is further intended to be used in conjunction with the container assembly 305 and they together constitute a refill for use with the wick diffuser 300. To secure the shroud assembly 306 to the container assembly 305 for usage of the wick diffuser 300, as shown in the exploded view of FIG. 62a, the shroud assembly can be slid within the opening 446 of the plug 420, until the stem 494 of the outer shroud member contacts and locks with the retention members 466 on the inner rim 444 of the plug, thereby rupturing the membrane 450 and enabling contact of the wick 308 with the VOC within the bottle 418 (or the bottle 424). It will be understood that for the threaded bottle 424, the bottle cap 320 is first released from the bottle before assembling the shroud assembly 306 thereto. An assembled view of the refill is shown in FIG. 62b, while a cross-sectional view thereof is shown in FIG. 60.

Furthermore, in at least some embodiments, a venting hole (not shown) on the top of the bottle 418 may provide venting to the VOC stored therein to prevent splashing of the VOC from within the bottle after the wick diffuser 300 has been activated. In other embodiments, wiping or sealing ribs to seal the pair of wick holders 322 to the outer shroud 324 and the wick 308 to the pair of wick holders, to prevent splashing of the VOC from the bottle 418 may be employed. In yet other embodiments, a window molded on a floor portion (e.g., the cut out on the bottom surface 487) of the pair of wick holders 322 may also prevent the VOC from leaking in between the pair of wick holders and the outer shroud 324. Other features to prevent splashing and leaking of VOC from the bottle 418 may be employed in other embodiments.

FIG. 60 in particular shows the container assembly 305 having the bottle 418 with the plug 420 securely assembled over the neck portion 430 thereof, as well as the shroud assembly 306 assembled with respect to the container assembly. As also shown, the wick 308 is secured to the shroud assembly 306, which in turn is shown to have the pair of wick holders 322 sandwiched between the outer shroud member 324. As described above, the container assembly 305 and the shroud assembly 306 have various retention features, which provide a one-way assembly such that once the refill is assembled, it is activated and any disassembly of the aforementioned parts or reuse of those parts is avoided by the user.

The various retention features described above are summarized below for clarity: (a) shroud to plug retention 496, which involves the retention of the shroud assembly 306 to the plug 420 via the retention members 466 and it can be user activated when activation of the wick diffuser 300 is required; (b) bottle to plug retention 498, which can typically be assembled during manufacture of the container assembly 305 and prevents the user from refilling the liquid within the bottle 418; and (c) shroud to wick retainer retention 500, which is the retention of the pair of wick holders 322 to the outer shroud member 324 and can also be assembled during manufacture of the unit to prevent re-use of the wick 308.

In addition to the various retention features, several seals and sealing surfaces are provided between the various members of the shroud assembly 306 and the container assembly 305 for preventing a rapid release of fragrance (or otherwise any leakage or spillage of the liquid) if the wick diffuser 300 is tipped. A first seal is a wick retainer to shroud seal 502, which as the name implies, is a seal between the pair of wick holders 322 and the outer shroud member 324. A second seal is a shroud to plug seal 504, which is a seal between the outer shroud member 324 and the plug 420, when the outer shroud member is positioned within the plug. A third seal is a plug to bottle seal 506 that exists between the inner rim 444 of the plug 420 and the neck portion 430 of the bottle 418. A fourth seal is a wick to wick retainer seal 508 formed between the wick 308 and the pair of wick holders 322 when the wick is retained between the pair of wick holders. By virtue of providing the aforementioned seals, the wick diffuser 300 can be made substantially spill-proof and any rapid diffusion of fragrance or leakage if liquid can be avoided.

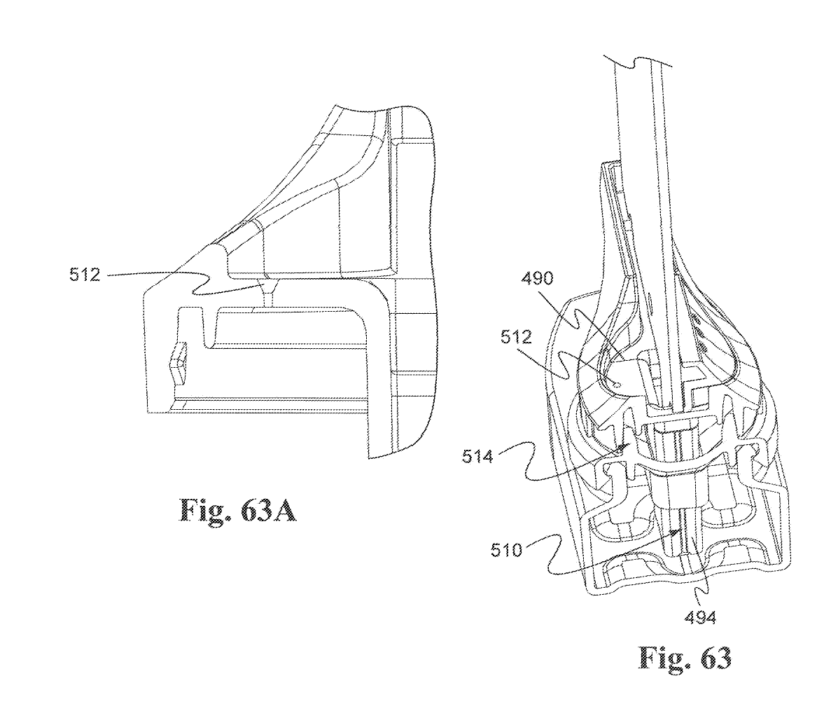

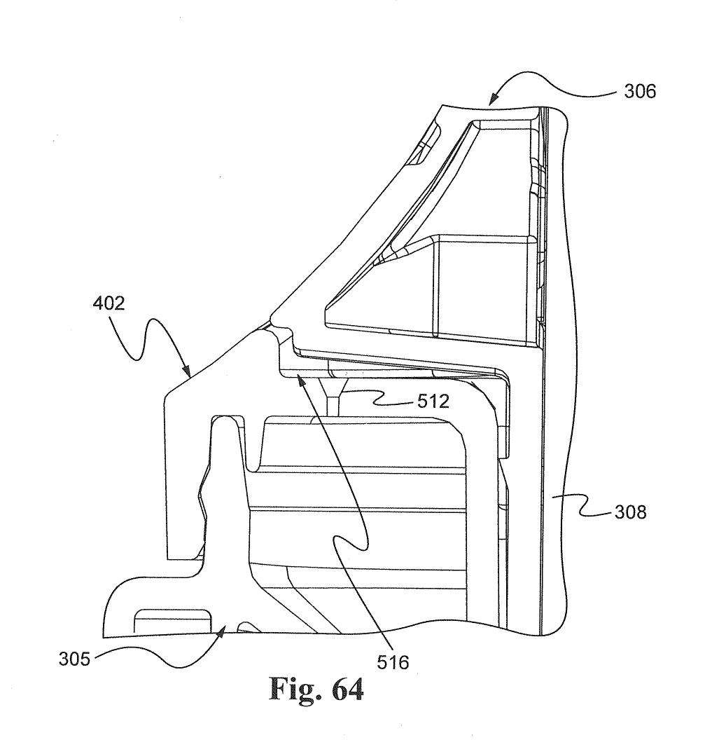

Referring now to FIGS. 63, 63a and 64, a venting feature formed within the wick diffuser 300 is shown in cut-away, in accordance with at least some embodiments of the present disclosure. To facilitate an upwardly diffusion of fragrance from the container assembly 305 through the wick 308, a pair of venting channels 510 can be provided within the wick diffuser 300. In particular, the venting channels 510 can be formed longitudinally along the stem 494 of the outer shroud member 324 and located diametrically opposite from one another. The venting channels 510 can lead to a pair of vent holes 512, which can be formed at a transverse angle (e.g., ninety degrees) to the venting channels and can be located on the lateral body section 490 of the outer shroud member 324. For improved moldability and efficiency of the venting channels 510, each of the vent holes 512 can be funnel-shaped, as shown in FIG. 63a.

Furthermore, the venting channels 510 can be connected to the vent holes 512 by way of an open chamber 514 formed between the plug 420 and the shroud assembly 306. Each of the pair of venting channels 510, the vent holes 512 and the open chamber 514 can form a vent path together with a second chamber 516 for dispersal of fragrance. The second chamber 516, as shown in FIG. 64 can be a channel formed at the interface of the pair of wick holders 322 and the outer shroud member 324 such that the wick holders bottom out at the edges of the outer shroud member, thereby ensuring a gap at the site of the vent holes 512. It will be understood that the vent holes 512 are only functional once the membrane 450 of the plug 420 has been ruptured and the shroud assembly 306 positioned within the container assembly 305.

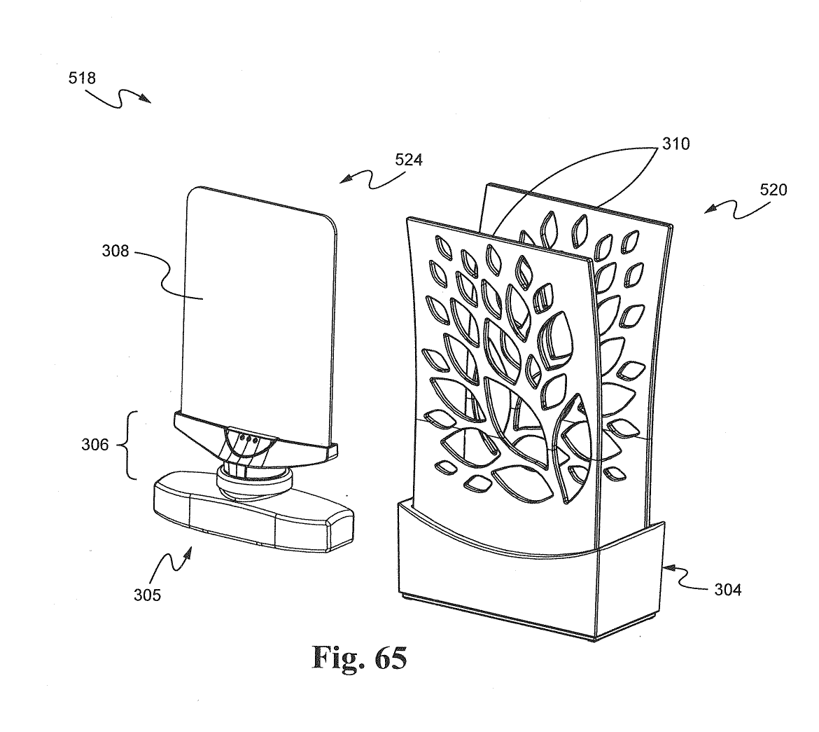

Turning now to FIG. 65, a starter kit 518 is shown, in accordance with at least some embodiments of the present disclosure. As shown, the starter kit 518 can include a first piece 520 having the base 304 along with the pair of panels 310. Although shown in FIG. 65 as assembled, it will be understood that in at least some embodiments, the panels 310 can come unassembled within the package for assembly to the base 304 by the user. Also included with the first piece 520 is a refill 524. The refill 524 can include the wick 308, the shroud assembly 306 and the container assembly 305. Notwithstanding the fact that the shroud assembly 306, the wick 308 and the container assembly 305 have been shown in an assembled configuration, it will be understood that typically, each of these components will be unassembled within the package for assembly by the user. It will also be understood that in at least some embodiments, the wick 308 can be pre-assembled with the shroud assembly 306. In some other embodiments, the various components of the shroud assembly 306 can come unassembled as well. Furthermore, upon exhaustion of the refill 524 provided along with the starter kit 518, a separate refill can be purchased for use the first piece 520.

### INDUSTRIAL APPLICABILITY

From the foregoing, it can be seen that the present disclosure sets forth a wick diffuser providing an enhanced aesthetic appeal as well as environmental friendliness. The wick diffuser includes the use of a planar shaped, and cellulosically formed center wick laterally flanked by first and second arcuate panels. The use of such a configuration is not only aesthetic, but also provides enhanced spill resistance in that if the wick diffuser is ever toppled, the arcuate panels hold the wick above horizontal and out of engagement with the arcuate panels themselves.

Moreover, the diffuser may be provided with a sealed container of VOC which is only exposed upon a user inserting a stem of the cellulosic wick. In order to facilitate such penetration, the stem may be enveloped by a protective shroud which reduces the insertion force needed to be applied by the user. This works in concert with the end of the protective shroud and/or the container seal being manufactured at a compound angle so as to ensure a single point of contact when the user insert the wick.

While only certain embodiments have been set forth, alternatives and modifications will be apparent from the above description to those skilled in the art. These and other alternatives are considered equivalents and within the spirit and scope of this disclosure and the appended claims.

What is claimed is:

1) A wick diffuser comprising:
a base;
at least one panel, the panel receivable within the base;
a container assembly positionable within the base and housing a liquid therewithin; and
a wick securable to the container assembly for dispersing fragrance from the liquid housed within the container assembly into a surrounding area, the wick being substantially planar in shape and including a body portion and a stem portion.

2) The wick diffuser of claim 1, wherein the base further comprises:
a first pair of wall members;
a second pair of wall members connected to the first pair of wall members to form an enclosed structure; and
a floor member having a top surface for supporting the first pair and the second pair of walls, and further having a first set of features for retaining the at least one panel, a second set of features for supporting the container assembly and a well for containing any spillage from the wick and the container assembly.

3) The wick diffuser of claim 2, wherein the first set of features comprises:
at least one pair of spring finger members, at least one of the at least one pair of spring finger members on each side of the first pair of wall members; and
at least two pairs of keying ribs, at least one pair of the at least two pairs of keying ribs on each side of the first pair of wall members and offset from one another, each of the at least two pairs of keying ribs extending upwardly from a top surface of the well.

4) The wick diffuser of claim 2, wherein the second set of features comprises:
a pair of ribs formed on the top surface of the floor member, each of the pair of ribs having a tapered configuration.

5) The wick diffuser of claim 2, wherein a bottom surface of the floor member further comprises two pairs of feet for supporting the base.

6) The wick diffuser of claim 1, wherein the base further comprises;
a base structure; and
a chassis capable of being received within the base structure, the chassis having at least two pairs of resilient cam members for retaining the chassis within the base structure, at least one pair of spring finger members and at least two pairs of keying ribs for retaining the at least one panel within the base.

7) The wick diffuser of claim 1, wherein the at least one panel further comprises:
a first panel member; and
a second panel member, each of the first and the second panel members having an arcuate configuration extending away from the base and a cut-out pattern on a top surface thereof for facilitating fragrance evaporation.

8) The wick diffuser of claim 7, wherein each of the first and the second panel members have at least one slot on a bottom portion for securing the first and the second panel members within the base.

9) The wick diffuser of claim 1, wherein the container assembly further comprises:
a bottle having (a) a body portion defining a cavity therewithin for housing the liquid; (b) a floor having a tapered configuration for directing the liquid toward a center portion of the bottle; and (c) a neck portion having a first pair of lugs;
a plug having (a) an outer rim for securing the plug to the neck portion of the bottle; and (b) an inner rim for receiving the wick and an additional structure and leading to a stem, the outer rim further having a second pair of lugs for locking with the first pair of lugs and the inner rim further having a pair of retention members for securing the additional structure.

10) The wick diffuser of claim 9, wherein the bottle can either be a threaded bottle or a non-threaded bottle.

11) The wick diffuser of claim 9, wherein the stem of the plug comprises a membrane formed at a compound angle for facilitating assembly of the wick and the additional structure within the container assembly, the membrane being a rupturable membrane with a coined seal.

12) The wick diffuser of claim 1, further comprising a shroud assembly for retaining the wick, the shroud assembly capable of being retained within the container assembly, and further comprising:
a pair of wick holders, each of the wick holders including a lateral body portion having a plurality of holes and posts for securing the pair of wick holders to the wick, and a stem leading transversely downwardly from the lateral body portion for receiving the stem portion of the wick and further having first and second retention members; and
a shroud having a lateral portion and a stem portion for securing the pair of wick holders to the shroud via the first and the second retention members.

13) The wick diffuser of claim 12, wherein a bottom portion of the wick comprises a plurality of holes and notches that mate with the plurality of posts and holes of the pair of wick holders.

14) The wick diffuser of claim 12, wherein the shroud defines a pair of venting channels along the stem portion thereof, and leading to a pair of vent holes via first and second chambers formed between the shroud and the container assembly, and the shroud and the pair of wick holders, respectively.

15) A wick diffuser comprising:
a base;
a container assembly positionable within the base;
a wick;
a shroud assembly having a pair of wick holders and a shroud for securing the wick in position, the shroud assembly securable within the container assembly; and
a pair of arcuate panel members securable within the base and extending upward and away from the base.

16) The wick diffuser of claim 15, wherein the wick is planar in shape and further comprises a body portion and a stem portion, the stem portion having a pair of holes and a pair of slots for attaching the wick to the pair of wick holders to form a first assembly.

17) The wick diffuser of claim 16, wherein the first assembly is secured to the shroud by inserting and locking the pair of wick holders to the shroud.

18) A wick diffuser kit comprising:
a first piece having a base and a pair of panel members; and
a refill having (a) a container assembly having a bottle housing a liquid and a plug for preventing access of the liquid by a user; (b) a shroud assembly having a pair of wick holders and a shroud, the shroud assembly capable of being assembled to the container assembly; and (c) a wick capable of being secured to the shroud assembly, the refill capable of being used with the first piece.

19) The wick diffuser kit of claim of claim 18, wherein the refill is disposable.

20) The wick diffuser kit of claim 19, wherein the pair of panel members are assembled to the base and the refill is positioned within the base when use of the wick diffuser is desired.
